(19) United States
(12) Patent Application Publication
(10) Pub. No.: US 2021/0332392 A1
(43) Pub. Date: Oct. 28, 2021

(54) COMPOSITIONS AND METHODS OF USE THEREOF FOR SCANDIUM SEPARATION FROM RARE EARTH CONTAINING MATERIAL

(71) Applicant: Lawrence Livermore National Security, LLC, Livermore, CA (US)

(72) Inventors: Dan Mcfarland Park, Dublin, CA (US); Ziye Dong, Dublin, CA (US); Yongqin Jiao, Pleasanton, CA (US)

(21) Appl. No.: 17/240,794

(22) Filed: Apr. 26, 2021

Related U.S. Application Data
(60) Provisional application No. 63/015,354, filed on Apr. 24, 2020.

(51) Int. Cl. C12P 3/00; C22B 61/00; C22B 3/18

(52) U.S. Cl. CPC C12P 3/00; C22B 3/18; C22B 61/00

(57) ABSTRACT

This disclosure provides microbes for the preferential separation of Scandium (Sc) from rare earth element (REE) containing materials, as well as methods of use thereof.

### PRIORITY CLAIM

This application claims priority to U.S. Provisional Application No. 63/015,354 filed on Apr. 24, 2020, the entire contents of each of which are incorporated herein by reference and relied upon.

### STATEMENT REGARDING FEDERALLY SPONSORED RESEARCH

The United States Government has rights in this application pursuant to Contract No. DEFWP-LLNL-18-FEW0239 between the United States Department of Energy, Office of Fossil Energy DE-NETL Rare Earth Program and Lawrence Livermore National Security, LLC for the operation of Lawrence Livermore National Laboratory.

### BACKGROUND

Scandium (Sc) is a high value transition metal (˜5000 US$/kg as scandium oxide, 99.9% purity) that is officially defined as a rare earth element (REE), along with the lanthanides and Yttrium. Scandium has many industrial applications, including Al—Sc alloys, solid oxide fuel cells, halide lamps, optics, catalyst ceramics, and lasers [3-5]. In particular, Al—Sc alloys are super-strong and light-weight, and have the potential to revolutionize the aerospace and automotive industries by enabling lighter and more fuel-efficient aircraft and vehicles [2].

However, the absence of reliable, secure, stable and long-term Sc production currently limits commercial applications of Sc. Furthermore, the majority of global Sc production (˜15 tonnes annually) comes from China and Russia, raising geopolitical concerns about the diversity of the Sc supply. As such there is a need to identify and exploit new sources of Sc.

Like REEs, Sc is not rare in its distribution across the earth's crust 11-31. However, Sc-rich minerals deposits rarely exceed a couple hundred ppm [1, 2]. Although Sc has a +3 charge like REEs, its significantly smaller ionic radius results in distinct geochemical behavior; many REE-enriched deposits lack relevant Sc concentrations [1, 2]. Indeed, there are currently no known economically viable, large-scale Sc resources in US or Europe [2]. However, there is an abundance of Sc-enriched waste products that represent potential Sc sources. This includes bauxite residue (i.e., red mud), generated at an annual production rate of 120 million tonnes as a byproduct of industrial alumina production and containing average Sc concentrations of 40-170 ppm [6], and coal/coal combustion products, generated at an annual production rate of 115 million tonnes/year (for CCP) in the US alone and containing average Sc concentrations of 36-70 ppm [7]. Both waste residues exhibit high matrix complexity, containing Fe, Al, Ca, Mg, Na at orders of magnitude higher concentration than Sc. Furthermore, the abundance of REEs in both feedstocks, necessitates a means to separate Sc from chemically similar REEs. While these waste residues have received significant recent attention as potential sources of critical REEs, technoeconomic analysis suggest that Sc separation is critical for the economic recovery of REEs, representing greater than 90% of the REE value 18-101.

### SUMMARY

Methods and materials are provided for the preferential separation of Sc from REE-containing materials.

In some aspects, the present disclosure provides a method for preferentially separating Sc from a REE containing material comprising the steps of: (a) contacting microbes with the REE containing material at a pH between about 3 to about 4 to form Sc-microbe complexes; and (b) separating the Sc from the microbes by contacting the Sc-microbe complexes with a solution comprising an organic chelator, wherein the microbes are A. nicotianae microbes. In some embodiments, in the contacting step (a) Sc is selectively absorbed by the microbes to form the Sc-microbe complexes and the microbes absorb substantially no other REEs, non-REE components, or any other elements in the REE containing material other than Sc. In some embodiments, the method further comprises repeating steps (a) and (b) with a second, third, fourth, fifth, six, seventh, eighth, ninth, tenth or more REE containing material.

In some embodiments, the organic chelator is citrate. In some embodiments, the solution comprises citrate at a concentration of about 25 mM. In some embodiments, solution comprising the organic chelator has a pH of about 5 to about 6. In some embodiments, the pH of the REE containing material is incrementally adjusted from a pH of about 3 to about 4 in the contacting step (a). In some embodiments, the pH of the REE containing material is incrementally adjusted from 3 to 3.4, 3.4 to 3.6, and 3.6 to 3.8 in the contacting step (a). In yet another embodiment, the solution is incrementally adjusted from pH 5 to 6 in the separating step (b). In some embodiments, the method further comprises adding the microbes to a column prior to step (a).

In some embodiments, step (b) is repeated until at least about 100%, at least about 90%, at least about 80%, at least about 70%, at least about 60%, at least about 50%, at least about 40%, at least about 30%, at least about 20%, or at least about 10% of the Sc is separated from the Sc-microbe complexes. In some embodiments, the Sc is separated relative to any other REE, any non-REE component, and/or to any other element in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE, any non-REE component, or any other element.

In another aspect, the present disclosure provides a method for preparing a particle for Sc separation from REE containing material comprising the steps of: (a) encapsulating A. nicotianae microbes in a nanoparticle to from microbe encapsulated particles; (b) selecting microbe encapsulated particles having an average size of about 150 μm to about 300 μm, wherein the microbes are embedded within or on a surface of the particles. In some embodiments, the nanoparticle is a silica nanoparticle. In yet another embodiment, the encapsulating step (a) includes a condensation reaction of SNPS with TEOS to form a microbe encapsulated gel. In some embodiments, prior to step (b), the microbe encapsulated particles are crushed to obtain particles having length in at least one dimension between about 150 μm to about 300 μm. In some embodiments, the method further comprises incorporating the particle into a column, membrane, bead, or combination thereof.

In yet another aspect, the present disclosure provides a particle for Sc separation comprising A. nicotianae, wherein the particle has an average pore size of about 50 nm to about 200 nm.

In some embodiments, the particle has a cuboid shape. In yet another embodiment, the particle has a length in all four dimensions between about 150 μm to about 300 μm. In another embodiment, the pore size facilitates the diffusion of REEs into and out of the particle. In some embodiments, the pore size prevents the diffusion of A. nicotianae cocci having an average diameter of at least 1 μm from diffusing into and out of the particle. In some embodiments, the particle has an A. nicotianae cell density of 1 g/ml. In some embodiments, the A. nicotianae cell density is at least about 20 wt % or more of the total weight of the particle or at least about 20 vol % or more of the total volume of the particle.

In another aspect, the present disclosure provides a method for preferentially separating Sc and total REEs from a REE containing material comprising the steps of: (a) contacting microbes embedded within a first solid support with the REE containing material at a pH of about 3 to about 4 to form Sc-microbe complexes; (b) collecting the REE containing material, wherein the REE material contains substantially no Sc after contact with the microbes embedded within the first solid support; and (c) contacting microbes embedded within a second solid support with REE material containing substantially no Sc to form REE-microbe complexes. In some embodiments, prior to the collecting step (b), Sc is separated from the microbes by contacting the Sc-microbe complex with a solution comprising an organic chelator. In some embodiments, after the contacting step (c), the total REEs are separated from the microbes by contacting the REE-microbe complexes with a solution comprising the organic chelator.

In yet another embodiment, after the contacting step (c), the total REEs are separated from the microbes by contacting the REE-microbe complexes with solution comprises HCl. In some embodiments, the solution has a pH of 1. In some embodiments, the organic chelator is citrate. In yet another embodiment, the solution has a pH of about 6. In some embodiments, prior to the contacting step (c), the pH of the REE containing material containing substantially no Sc is adjusted to about 5 to precipitate non-REE components from the REE containing material, wherein the precipitated non-REEs are filtered from the REE containing material.

In some embodiments, the other REEs are selected from the group consisting of La, Ce, Pr, Nd, Pm, Sm, Eu, Gd, Tb, Dy, Ho, Er, Tm, Yb, Lu, and Y. In yet another embodiment, the non-REE component is a metal selected from the group consisting of Fe, Ca, Al, Mg, Zn, Ni, Na, Li, K, Co, Mn, and Cu. In some embodiments, the non-REE component is a radionucleotide selected from the group consisting of U and Th.

In some embodiments, the microbes are embedded into a solid support. In some embodiments, the microbes are embedded into SiO₂. In yet another embodiment, a cell density of the microbes in the SiO₂ is about 1 g/ml. In some embodiments, a cell density of the microbes in the SiO₂ is about 2 g/ml.

In some embodiments, Sc is preferentially separated from Fe in the REE containing material. In some embodiments, the Fe and/or Al are present in the REE containing material in a concentration three orders of magnitude higher than that of a concentration of Sc. In some embodiments, the microbes selectively bind to the Sc due to a stronger ionic interaction of Sc relative to other REEs or non-REE components.

In some embodiments, the microbes are A. nicotianae.

In another aspect, the present disclosure provides a method for preferentially separating one or more rare earth elements (REEs) from an REE containing material comprising the steps of: (a) contacting microbes with the REE containing material to form REE-microbe complexes, wherein the microbes are encapsulated in a polyethylene glycol diacrylate hydrogel; and (b) separating the one or more REEs from the microbes by contacting the REE-microbe complexes with a solution comprising an organic chelator.

In some embodiments, the one or more REEs are selected from the group consisting of La, Ce, Pr, Nd, Pm, Sm, Eu, Gd, Tb, Dy, Ho, Er, Tm, Yb, Lu, Sc, and Y. In some embodiments, the one or more REEs is Sc.

In some embodiments, the polyethylene glycol diacrylate hydrogel encapsulated microbes are in a form of a nanoparticle having a having an average size of about 150 μm to about 700 μm. In some embodiments, the average size is about 300 μm to about 500 μm. In some embodiments, the average size is the average size is about 150 μm to about 300 μm. In some embodiments, the average is size about 500 μm to about 700 μm.

In some embodiments, the method further comprises adding the microbes to a column prior to step (a). In some embodiments, contacting the microbes with the REE containing material comprises introducing the REE containing material to the column at a flow rate of about 2×10⁻³ m/s to 4×10⁻³ meters per second (m/s).

In some embodiments, the REE containing material comprises the one or more REEs at a concentration of about 1.0 mM to about 3.0 mM. In yet another embodiment, the concentration is about 2.2 mM.

In some embodiments, the one or more REEs is Sc and in the contacting step (a) Sc is selectively absorbed by the microbes to form the Sc-microbe complexes and the microbes absorb substantially no other REEs, non-REE components, or any other elements in the REE containing material other than Sc.

In some embodiments, step (b) is repeated until at least about 100%, at least about 90%, at least about 80%, at least about 70%, at least about 60%, at least about 50%, at least about 40%, at least about 30%, at least about 20%, or at least about 10% of the one or more REEs is separated from the REE-microbe complexes.

In some embodiments, the one or more REEs is separated relative to any other REE, any non-REE component, and/or to any other element in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE, any non-REE component, or any other element.

In some embodiments, the method further comprises repeating steps (a) and (b) with a second, third, fourth, fifth, six, seventh, eighth, ninth, tenth or more REE containing material.

In another aspect, the present disclosure provides a method for preferentially separating scandium (Sc) from a REE containing material comprising the steps of: (a) adding microbes embedded within polyethylene glycol diacrylate hydrogel to a column; (b) introducing to the microbes embedded within polyethylene glycol diacrylate hydrogel the REE containing material at a flow rate of about 2×10-3 m/s to 4×10-3 meters per second (m/s) and at a pH of about 3 to about 4 to form Sc-microbe complexes; and (c) separating the Sc from the microbes by contacting the Sc-microbe complexes with a solution comprising an organic chelator.

In some embodiments, the solution has a pH of about 6. In some embodiments, the solution comprising the organic chelator has a pH of about 5 to about 6. In some embodiments, the organic chelator is citrate. In some embodiments, the solution comprises citrate at a concentration of about 25 mM.

In some embodiments, the Sc is present in the REE containing material at a concentration of about 1 μM to about 3 mM. In some embodiments, Sc is present in the REE containing material at a concentration of about 2 mM.

In yet another embodiment, a pH of the REE containing material is incrementally adjusted from a pH of about 3 to about 4 in the contacting step (a). In another embodiment, a pH of the REE containing material is incrementally adjusted from 3 to 3.4, 3.4 to 3.6, and 3.6 to 3.8 in the contacting step (a). In some embodiments, the solution is incrementally adjusted from pH 5 to 6 in the separating step (b) or step (c).

In some embodiments, the other REEs are selected from the group consisting of La, Ce, Pr, Nd, Pm, Sm, Eu, Gd, Tb, Dy, Ho, Er, Tm, Yb, Lu, and Y. In yet another embodiment, the non-REE component is a metal selected from the group consisting of Fe, Ca, Al, Mg, Zn, Ni, Na, Li, K, Co, Mn, and Cu. In some embodiments, the non-REE component is a radionucleotide selected from the group consisting of U and Th.

In another aspect, the present disclosure provides a method for preparing a particle for separation one or more rare earth elements (REEs) from REE containing material comprising the steps of: (a) encapsulating microbes in a polyethylene glycol diacrylate hydrogel to from microbe encapsulated particles; and (b) selecting microbe encapsulated particles having an average size of about 300 μm to about 500 μm, wherein the microbes are embedded within or on a surface of the particles.

In some embodiments, the microbes are encapsulated in a polyethylene glycol diacrylate hydrogel by free radical polymerization of polyethylene glycol diacrylate. In some embodiments, prior to step (b), the microbe encapsulated particles are crushed to obtain particles having an average size of about 150 μm to about 700 μm. In another embodiment, the method further comprises selecting microbe encapsulated particles having an average size of about 300 μm to about 500 μm from the particles having an average size of about 150 μm to about 700 μm.

In some embodiments, the one or more REEs are selected from the group consisting of La, Ce, Pr, Nd, Pm, Sm, Eu, Gd, Tb, Dy, Ho, Er, Tm, Yb, Lu, Sc, and Y. In some embodiments, the one or more REEs is Sc.

In some embodiments, the method further comprises incorporating the particle into a column, membrane, bead, or combination thereof.

In some aspects, the present disclosure provides a particle for separation of one or more rare earth elements (REEs) comprising Arthrobacter nicotianae (A. nicotianae) encapsulated in a polyethylene glycol diacrylate hydrogel, wherein the particle has an average size of about 300 μm to about 500 μm.

In some embodiments, the particle has a cuboid shape.

In some embodiments, the particle has an A. nicotianae cell density of 1 g/ml. In yet another embodiment, A. nicotianae cell density is at least about 20 wt % or more of the total weight of the particle or at least about 20 vol % or more of the total volume of the particle.

### BRIEF DESCRIPTION OF THE DRAWINGS

FIG. 1 shows the distribution coefficients for each REE following biosorption assays with A. nicotianae in a synthetic solution containing equimolar concentrations of individual REEs in accordance with embodiments of the present disclosure. Error bars were calculated using a formula for error propagation [5] from triplicate samples.

FIGS. 2A-2B are representative plots showing that aluminum (Al) precludes high REE recovery efficiencies at pH 4 in accordance with embodiments of the present disclosure. FIG. 2A shows the total REE recovery efficiency by LBT-displayed E. coli and native A. nicotianae in pH 4 lignite leachate. FIG. 2B shows the metal composition (mg/L) in the solutions following a biosorption/desorption cycle plotted over a range of cell densities for pH 4 adjusted lignite for A. nicotianae. The REE recovery efficiencies from FIG. 3 are replotted as blue dots as a reference.

FIGS. 3A-3B are representative plots showing extraction of scandium (Sc) at pH 4 from lignite leachate in accordance with embodiments of the present disclosure. FIG. 3A shows that fraction of individual metals recovered from lignite leachate (pH 4) over a range of A. nicotianae cell densities. FIG. 3B shows the separation factor for Sc relative to select metals (Mₓ) in lignite leachate for LBT displayed—E. coli and A. nicotianae. Data are depicted as the log transformed SF_{Sc,M} values, with values greater than zero indicative of enhanced selectivity for Sc relative to Mₓ.

FIG. 4 are representative plots showing the selective extraction of Sc from pH 4 lignite leachate in accordance with embodiments of the present disclosure. The plots show the fraction of individual metals recovered from lignite leachate (pH 4) over a range of LBT-displayed E. coli cell densities.

FIG. 5 is a representative schematic showing microbe encapsulated SiO₂ gel (MESG) particle fabrication in accordance with embodiments of the present disclosure.

FIGS. 6A-6D are representative images of the microbe encapsulated in SiO₂ gel in accordance with embodiments of the present disclosure. FIGS. 6A and 6B are SEM and TEM images, respectively and FIGS. 6C-6D show images of the microbe encapsulated in SiO₂ gel.

FIGS. 7A-7B are representative confocal images of the microbe encapsulated SiO₂ gel in accordance with embodiments of the present disclosure.

FIGS. 8A-BC are representative plots showing the batch Sc sorption in accordance with embodiments of the present disclosure. FIG. 8A is a Langmuir isotherm showing the variation of adsorption against the equilibrium concentration for adsorption of Sc on silica gels. FIG. 8B shows the maximum Sc adsorption amount calculated according to Langmuir isotherm. Lastly, FIG. 8C shows the fractional Sc desorbed by variation of citrate concentrations.

FIG. 9A-9B are representative plots shows adsorption of and desorption kinetics of batch Sc in accordance with embodiments of the present disclosure. FIG. 9A shows the adsorption and FIG. 9B shows the desorption kinetics on no cell control, 0.5 g/mL, 1.0 g/mL, and 2 g/mL microbe encapsulated silica gels.

FIGS. 10A-10C are representative plots showing Sc adsorption in a fixed-bed column with microbe encapsulated silica gels in accordance with embodiments of the present disclosure. FIG. 10A shows the breakthrough curves of no cell control and 1.0 g/mL microbe encapsulated silica gels (Feed solution: 0.7 mM Sc, 10 mM glycine, pH 3). FIG. 10B shows the adsorption capacity calculated for adsorbent. Lastly, FIG. 10C shows the effect of citrate concentration on Sc desorption curves (1.0 g/mL gel).

FIGS. 11A-11B are representative plots showing column reusability in accordance with embodiments of the present disclosure. FIG. 11A shows the Sc breakthrough curves for each of 10 consecutive adsorption/desorption cycles at a flow rate of 1 mL/min. The column was reconditioned by 10 mL 10 mM pH 3 glycine between each cycle. FIG. 11B shows column adsorption capacity calculated for each cycle by using mass balance in accordance with embodiments of the present disclosure.

FIGS. 12A-12B are representative plots showing the breakthrough curves for metal ions in the synthetic solution and desorption profiles of metal ions, respectively in accordance with embodiments of the present disclosure.

FIGS. 13A-13B are representative plots showing breakthrough curves for major metal ions in the lignite leachate and comparison of metal ions composition between lignite solution and synthetic solution, respectively in accordance with embodiments of the present disclosure.

FIGS. 14A-14B are representative plots showing breakthrough curves for Sc and Fe in lignite solutions with different pH adjustment, respectively in accordance with embodiments of the present disclosure.

FIGS. 15A-15C are representative plots showing a comparison of lignite composition before and after pH adjustment (FIG. 15A); breakthrough curves for metal ions in the pH adjusted lignite leachate (FIG. 15B); and desorption profiles of metal ions (FIG. 15C) in accordance with embodiments of the present disclosure.

FIG. 16 is a schematic of a process flow diagram for biosorption-based REE recovery of Sc and total REEs from coal and coal byproducts in accordance with embodiments of the present disclosure.

FIG. 17 is a schematic showing a two-stage packed-bed bioreactor design for sequential Sc and REE+Y recovery from coal byproduct feedstock in accordance with embodiments of the present disclosure.

FIGS. 18A-18B are representative plots showing distribution coefficients (Kd) (FIG. 18A) and separation factors (FIG. 18B) of non-encapsulated A. nicotianae for Al, Sc, Fe, Y, and Nd in 10 mM glycine at pH 3 in accordance with an embodiment of the present disclosure.

FIG. 19 is a representative plot showing Sc selectivity of MESG, where the separation factor for Sc relative to select REEs and Non-REEs was determined by exposing the MESG biosorbent (1.0 g/mL cell loading) to a multi-element solution (Sc, Fe(III), Al, Nd, Y) in accordance with embodiments of the present disclosure.

FIGS. 20A-20B are representative plots showing distribution coefficients (Kd) (FIG. 20A) and separation factors (FIG. 20B) of cell-free silica for Al, Sc, Fe, Y, and Nd in 10 mM glycine at pH 3.0 in accordance with embodiments of the present disclosure.

FIGS. 21A-21C are representative plots showing batch Nd adsorption by MESG particles including a Langmuir isotherm showing the Nd adsorption capacity as a function of equilibrium Nd concentration at pH 3 and 5 (FIG. 21A), maximum Nd adsorption amount calculated according to Langmuir isotherm (FIG. 21B), and fraction of Nd desorbed at different citrate (pH 6) concentrations (FIG. 21C) in accordance with embodiments of the present disclosure.

FIGS. 22A-22B are representative plots showing distribution coefficients (Kd) of MESG for Al, Sc, Fe(III), Y, and Nd (FIG. 22A) or Al, Sc, Fe(II), Y, and Nd (B) in 10 mM glycine at pH 3 (FIG. 22B), (*) denotes that the adsorption of Al and Fe were below the detection limit in accordance with embodiments of the present disclosure.

FIG. 23 is a representative plot showing concentration ratio of each metal ion in the biosorption eluent (6 bed volumes) compared to the pH 3.4 lignite feed solution in accordance with embodiments of the present disclosure.

FIG. 24 is a representative schematic showing a fabrication process for microbe encapsulation in PEGDA gels in accordance with embodiments of the present disclosure.

FIGS. 25A-25B are representative images of microbes encapsulated in PEGDA gels in accordance with embodiments of the present disclosure. FIG. 25A is SEM image of microbes encapsulated in PEGDA and FIG. 25B is an enlarged image of FIG. 25A, showing the pores of microbe encapsulated PEGDA.

FIGS. 26A-26C are representative plots of Sc adsorption in an 18 mL fixed-bed column comprising microbes encapsulated in PEGDA in accordance with embodiments of the present disclosure. The plots show breakthrough curves of 150-300, 300-500, and 500-700 μm particles packed columns (Feed solution: 2.2 mM Sc, 10 mM glycine, pH 3.0) (FIG. 26A), breakthrough curves of a 300-500 μm particles packed column at different flow rates (Feed solution: 2.2 mM Sc, 10 mM glycine, pH 3.0) (FIG. 26B), and breakthrough curves of 300-500 μm particles packed column at different feed Sc concentrations (FIG. 26C). The data were fit to a Bohart-Adams model (solid line).

FIGS. 27A-27C include a representative schematic for a pure water flux experimental set up for calculation of pressure drops (FIG. 27A), a corresponding representative plot showing pure water flux obtained with different particle sizes of microbes encapsulated in PEGDA (FIG. 27B), and pressure drops calculated according to pure water flux experiments (FIG. 27C) in accordance with embodiments of the present disclosure.

FIGS. 28A-28B are representative plots showing the effect of citrate concentration on Sc desorption curves (FIG. 28A) and column reusability tests (FIG. 28B) in accordance with embodiments of the present disclosure.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of example only, and not limitation. As such, this detailed description of various alternative embodiments should not be construed to limit the scope or breadth of the present invention as set forth below.

Traditionally, an acid leaching process is the first step in the recovery of Sc from Sc bearing materials, followed by selective precipitation or a solvent extraction processes to produce a concentrated Sc product [11]. However, the low Sc concentration in waste feedstock leachates limits the efficacy of precipitation and solvent extraction for Sc recovery [12]. A precipitation step is expected to produce insufficiently pure Sc because of the co-precipitation of abundant non-REE metals, such as Fe and Al. On the other hand, the solvent extraction process raises not only economical but also environmental concerns as the loss of expensive and hazardous organic solvents increases when dealing with diluted feed solution.

Owing to the above limitations, solid-liquid extraction (SLE) has emerged for the recovery of Sc from dilute solution as an environmentally friendly alternative. In order to establish a feasible SLE process, the key is to develop adsorbent materials that can repeatedly adsorb and desorb Sc without substantial loss in capacity. So far, a number of adsorbents have been developed for Sc recovery, including polyelectrolytes, carbon-based materials, resins and silica [13-15]. Although these adsorbents have shown promising Sc adsorption capacity, they are limited by relatively low selectivity, resulting in low Sc purity. For example, it has been reported that Fe, Al, and Ca were also adsorbed by resin while co-removal of Al, Cu and Cr was reported by ligand grafted algae.

Microbe-mediated surface adsorption (biosorption) represents a potentially cost-effective and environmentally sustainable SLE approach for REE recovery from dilute solutions [16-19]. Microorganisms synthesize and display high-density surface-accessible functional groups (e.g., carboxylates and phosphates) during growth, facilitating high-capacity REE adsorption [19]. Adsorbed REEs can be readily recovered by desorption using water-soluble organic acids such as citrate [20], and the biomass can be reused, independent of cell viability [21]. In addition, preferential adsorption of REEs over most non-REEs by cell surface functional groups [22-25] has yielded promising results even with complex sample matrices such as leachate from a phosphor powder [26], NdBFe hard disk drive magnets [21, 27], mine tailings [28], and coal byproducts [29]. However, the efficacy of biosorption for selective Sc recovery from REE-enriched industrial feedstocks as complex as bauxite residue or coal/CCP leachate remains untested.

As disclosed herein, a cell encapsulation approach was developed in which Arthrobacter nicotianae (A. nicotianae) was embedded within a Si-sol gel matrix and the resulting microbe particles were used to make packed-bed columns. The results suggest that at pH 3, microbe particles enable selective extraction of Sc under flow through conditions with high column stability; greater than 95% of the adsorption capacity is retained over 10 adsorption/desorption cycles with Sc. The biosorption-based approach also shows that downstream REE extraction can be achieved with the Sc-depleted leachate following a pH 5 adjustment step. Importantly, this process enables a one-step separation of Sc from physiochemically similar REEs and enables downstream separation of total REEs from non-REEs using a second, higher pH biosorption step.

Accordingly, provided herein is a biosorption-based method for the selective recovery of Sc from low-grade, abundant waste products, including coal/coal byproducts and bauxite residues. Also provided herein are microbes for use in the disclosed methods. The use of microbes for preferentially separating Sc from REE-containing materials as described herein overcome the technical, economic, and environmental limitations of conventional Sc separation technologies.

### Definitions

The term “about” as used herein when referring to a measurable value such as an amount or concentration and the like, is meant to encompass variations of 20%, 10%, 5%, 1%, 0.5%, or even 0.1% of the specified amount.

The terms “acceptable,” “effective,” or “sufficient,” when used herein to describe the selection of any components, ranges, dose forms, etc., intend that said component, range, dose form, etc. is suitable for the disclosed purpose.

The terms “no” or “substantially no” as used herein with regard to a component of a material, composition, or solution mean that the component (e.g., a competing metal) is present in an amount less than about 0.0001%, less than about 0.001%, less than about 0.01%, less than about 0.1%, less than about 1%, less than about 5%, or less than about 10% of the total weight or volume of the material, composition, or eluted solution.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

As used herein, the singular forms “a,” “an,” and “the” include plural referents unless the context clearly dictates otherwise. Thus, for example, reference to “a microbe” includes a plurality of microbes.

### Microbes

Aspects of the disclosure provide microbes for use in separating REEs, including genetically engineered to express REE binding ligands, such as lanthanide binding tags (LBT). Suitable microbes, including suitable genetically modified microbes are descripted in US Publication No. 2018/0195147, which is incorporated by reference in its entirety.

Non-limiting examples of suitable bacteria include Acetobacter spp., Acidithiobacillus spp., Acinetobacter spp., Aeromonas spp., Agrobacterium spp., Alcaligenes spp., Archaebacteria spp., Aquaspirrilum spp., Arthrobacter spp., Azotobacter spp., Bacillus spp., Caulobacter spp., Chlamydia spp., Chromatium spp., Chromobacterium spp., Citrobacter spp., Clostridium spp., Comamonas spp., Corynebacterium spp., Cyanobacteria spp., Escherichia spp., Flavobacterium spp., Geobacillus spp., Geobacter spp., Gluconobacter spp., Lactobacillus spp., Lactococcus spp., Microlunatus spp., Mycobacterium spp., Pantoea spp., Pseudomonas spp., Ralstonia spp., Rhizobium spp., Rhodococcus spp., Saccharopolyspora spp., Salmonella spp., Serratia spp, Sinorhizobium spp., Stenotrophomonas spp., Sireptococcus spp., Streptomyces spp., Synechocystis spp., Thermus spp., Xanthomonas spp., and Zymonas spp.

In one embodiment the bacterium is selected from the group consisting of Caulobacter (e.g., C. crescentus, C. bacteroides, C. daechungensis, C. fusiformis, C. ginsengisoli, C. halobacteroides, C. henricii, C. intermedius, C. leidyi, C. maris, C. mirabilis, C. profindus, C. segnis, C. subvibrioides, C. variabilis, and C. vibrioides), Escherichia (e.g., E. albertii, E. coli, E. fergusonii, E. hermannii, and E. vulreris), Bacillus (e.g., B. licheniformis, B. cereus and B. subtilis), and Lactobacillus (e.g., L. lactis, L. acidophilus, L. brevis, L. bulgaricus, L. casei, L. helveticus, L. reuteri, L. rhamnosus, L. rhamnosus GG, L. rhamnosus GR-1. L. plantarum, and L. siliarius). In one preferred embodiment, the bacterium is C. crescentus. Caulobacter are particularly suitable because they are considered to be non-pathogenic, heavy metal resistant and oligotrophic. In another preferred embodiment, the bacterium is E. coli.

In some embodiments, the present disclosure provides microbes for use in separating one or more REEs, including Scandium (Sc), from REE containing materials, for example Arthrobacter nicotianae (A. nicotianae) microbes.

REEs are a group of seventeen chemical elements that includes yttrium and fifteen lanthanide elements. Sc is found in most REE deposits and is often included.

TABLE 1
Rare Earth Elements

| | | Atomic | | | Atomic |
|---|---|---|---|---|---|
| Name | Symbol | Number | Name | Symbol | Number |
| lanthanum | La | 57 | dysprosium | Dy | 66 |
| cerium | Ce | 58 | holmium | Ho | 67 |
| praseodymium | Pr | 59 | erbium | Er | 68 |
| neodymium | Nd | 60 | thulium | Tm | 69 |
| promethium | Pm | 61 | ytterbium | Yb | 70 |
| samarium | Sm | 62 | lutetium | Lu | 71 |
| europium | Eu | 63 | scandium | Sc | 21 |
| gadolinium | Gd | 64 | yttrium | Y | 39 |
| terbium | Tb | 65 | | | |

The microbes can bind to one or more REEs and preferentially separate the one or more REEs from other REEs and/or groups of REEs, for example, from lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), Scandium (Sc) or any combination thereof.

The microbes can bind to Sc and preferentially separate Sc from La, Ce, Pr, Nd, Pm, Sm, Eu. Gd, Tb, Dy, Ho, Er, Tm, Yb, Lu, Y, or any combination thereof.

In some embodiments, the microbes bind to a one or more REE ions with a binding affinity (K_{d}) between about 1 nM and 500 μM, about 100 nM and 200 μM, or about 500 nM and 1 μM. In some embodiments, the K_{d} is between about 500 nM and about 200 μM, about 1 μM and 200 μM, or about 50 μM and 100 μM. In some embodiments, the K_{d} is about 1 μM, about 5 μM, about 10 μM, about 15 μM, about 30 μM, about 40 μM, about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, about 200 μM, or more. In some embodiments, the K_{d} is in the μM range. In other embodiments, the K_{d} is in the nM range. In still other embodiments, the K_{d} is in the μM range. Affinity can be determined by any suitable means known to one of skill in the art. Non-limiting examples include, titration with REEs and detection using fluorescence, circular dichroism. NMR or calorimetry, inductively coupled plasma mass spectrometry, or spectroscopy. In the case of tightly binding sequences, it may be necessary to employ competition experiments.

In some embodiments, the microbes bind to a Sc ion with a binding affinity (K_{d}) between about 1 nM and 500 μM, about 100 nM and 200 μM, or about 500 nM and 1 μM. In some embodiments, the K_{d} is between about 500 nM and about 200 μM, about 1 μM and 200 μM, or about 50 μM and 100 μM. In some embodiments, the K_{d} is about 1 μM, about 5 μM, about 10 μM, about 15 μM, about 30 μM, about 40 μM, about 50 μM, about 60 μM, about 70 μM, about 80 μM, about 90 μM, about 100 μM, about 110 μM, about 120 μM, about 130 μM, about 140 μM, about 150 μM, about 160 μM, about 170 μM, about 180 μM, about 190 μM, about 200 μM, or more. In some embodiments, the K_{d} is in the μM range. In other embodiments, the K_{d} is in the nM range. In still other embodiments, the K_{d} is in the μM range. Affinity can be determined by any suitable means known to one of skill in the art. Non-limiting examples include, titration with Sc and detection using fluorescence, circular dichroism, NMR or calorimetry, inductively coupled plasma mass spectrometry, or spectroscopy. In the case of tightly binding sequences, it may be necessary to employ competition experiments.

In some embodiments, the microbes are related to Sc separation; however, the microbes of the present disclosure are similarly applicable to the separation of any REE including La, Ce, Pr, Nd, Pm, Sm, Eu, Gd, Tb, Dy, Ho, Er, Tm, Yb, Lu, Sc, and/or Y. For example, in some embodiments, the microbes can bind to one or more REEs and/or facilitate the separation of one or more REEs and/or groups of one or more REEs. The embodiments of the microbes are not limited to Sc separation.

### Biosorption Systems

Also provided are systems (i.e., biosorption/adsorption media) for REE extraction, including, but not limited to, Sc, and preferential separation comprising an amount of a genetically engineered microbes described herein, In some embodiments, the microbes are A. nicotianae microbes.

In some embodiments, the microbes are attached to a solid support, for example, a column, a membrane, a bead, or the like. The solid support can be any suitable composition known to one of skill in the art including, for example, a polymer, alginate, acrylamide, regenerated cellulose, cellulose ester, plastic, agarose, or glass.

These biosorption media, which include, for example, biofilm, microbe beads, and carbon nanotube embedded membranes can be used for adsorption under continuous flow. It is contemplated that microbe immobilization in biosorption media for use in flow through setups allows for complete (or substantially complete) separation of Sc and total REEs from REE-containing mixed metal solutions in a single step and, for example, without the need of centrifugation, filtration, or both.

In some embodiments, the disclosure provides composition comprising an amount of the microbes for example, A. nicotianae microbes.

In one embodiment, the microbes are immobilized via the formation of a biofilm. A biofilm is a layer of microorganisms that are attached to a surface. For biofilm formation, microbes having the distinctive ability to self-immobilize on supported solid surfaces.

Microbes can be immobilized on any suitable supporting material for optimal microbe attachment (e.g., fast, stable) known to one of skill in the art. Non-limiting examples of supporting material include carbon film, glass, steel, Teflon, polyethylene and the like. Growth media, temperature, inoculum size, incubation temperature, or any combination thereof can be varied to determine the optimal conditions for biofilm formation on each supporting material.

In one embodiment, the microbes are bound (i.e., embedded) within or to the surface of a bead. In some embodiments, the bead is a polymer. Suitable polymers include PEG (e.g., ˜10% PEG), alginate (e.g., ˜2% calcium alginate), agarose, and acrylamide (e.g., ˜10% polyacrylamide). In other embodiments the beads are glass, plastic, or steel.

In one embodiment, the microbes are immobilized through fabrication of micro beads. The synthesis and fabrication of micro bead in the 10 to 1000's microns size range for material encapsulation, storage and release have received significant attention in the past years for different applications, in order to isolate and protect the core materials from the surrounding environment. For example, encapsulation can protect enzymes from denaturing by solvents, shield probiotic bacteria from high temperature and digestive system, and protect chemicals from deteriorating due to oxidation and moisture with an inert matrix or shell. Moreover, encapsulations can allow and improve the controlled release of the encapsulated ingredient or immobilize living cells for controlled growth. As used herein, the term “encapsulate” is used interchangeable with the term “embed.”

The microbes can be provided in a reactor. Reactors can be configured in any suitable arrangement known to one of skill in the art, for example, spiral sheet and fiber brush, column purification, and filtration systems. Operation parameters and modeling that can be optimized by one of skill in the art include, for example, flow rate, extraction efficiency and product purification, solution conditioning (e.g., calcium addition), and surface complexation modeling (SCM) and performance optimization and prediction.

Biosorption is a chemical process based on a variety of mechanisms such as adsorption, absorption, ion exchange, surface complexation, and precipitation. When coupled with a material of biological origin such as microbes or biomass, this material is referred to as biosorption material. A biosorption material can for example, bind to Sc and separate Sc from REE containing materials (e.g., feedstocks). Provided herein are biosorption materials comprising microbes for preferentially separating Sc from REE containing material. The Sc extraction and preferential separation comprising an amount of the A. nicotianae microbes.

In some embodiments, the biosorption material is a bead and/or capsule. In some embodiments, the bead and/or capsule is suitable for the separation of Sc. In some embodiments, the biosorption material is a micro bead. As used herein, the term “microbe capsule” is used interchangeably with “microbe bead” and the term “capsule” is used interchangeably with “bead.”

Any suitable microencapsulation techniques known to one of skill in the art can be used to encapsulate the microbes of the present disclosure. In some embodiments, polymers such as acrylamide, silicone, and acrylate are used. Polymers have become the primary shell/matrix material used in this area because of the high solubility in aqueous media and/or organic solvents, easy and versatile formation, crosslinkable nature, sufficient strength and wide variety of chemistries.

In other embodiments, the disclosure provides methods of preparing a particle for Sc separation. In some embodiments, the methods for preparing a bead for REE separation comprise: (a) encapsulating A. nicotianae microbes in a nanoparticle to form microbe encapsulated particle; and (b) selecting microbe encapsulated particles having a length in at least one dimension between about 150 μm to about 300 μm; wherein the A. nicotianae microbes are embedded within or on a surface of the particles.

In some embodiments, the nanoparticle is comprised of polymeric material. In some embodiments, the polymeric material is acrylamide, silicone, and acrylate. In some embodiments, the polymeric material is silica nanoparticles (SNPs).

In some embodiments, the A. nicotianae microbes are embedded in a SNP. The microbes can be encapsulated in a crosslinked SNP matrix in a high cell density. In some embodiments, the SNP is crosslinked with silanes. Non-limiting examples of suitable silanes for crosslinking the SNP include tetramethyl orthosilicate (TMOS), triethoxymethylsilane (MTM), and 1,2-bis(triethoxysilyl)ethane (BTESE). In some embodiments, the A. nicotianae are embedded in the SNP by a condensation reaction with TEOS, TMOS, and/or MTM to form a microbe encapsulated silica gel.

In some embodiments, the solution comprised of SNP, A. nicotianae cells, and silane are mechanically mixed prior to formation of the gel. The SNP:Cell suspension can have a high viscosity that precludes homogenous mixing with the silanes using a microfluidic approach. In some embodiments, after mechanically mixing the SNP:Cell:slilane solution, the resulting gel-like solution is dried overnight (e.g., 24 h) to from the microbe encapsulated silica gel.

In some embodiments, the microbe encapsulated silica gel is crushed to form particles of various sizes. Crushing the microbe encapsulated gel can include pulverization and/or compression with force. The crushing step reduces the microbe encapsulated silica gel to fine particles. In some embodiments, the methods comprise selecting particles having an average size between about 150 μm to about 300 μm from the particles of various sizes. In some embodiments, after crushing the microbe encapsulated silica gel to form the crushed particles of various sizes, the crushed particles are passed through a sieve (e.g., a filter) that permits the separation of particles having an average size between about 150 μm to about 300 μm from the remainder of the particles. In some embodiments, the particles have average size in one and/or all dimensions of about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, or about 300 μm. In some embodiments, the particles have an average size in one and/or all dimensions between about 150 μm to about 200 μm, about 200 μm to about 300 μm, about 250 μm to about 300 μm, about 180 μm to about 300 μm, about 270 μm to about 300 μm, or about 160 μm to about 260 μm.

In other embodiments, the disclosure provides methods of preparing a particle for separation of one or more REE, including, but not limited to, Sc. In some embodiments, the methods for preparing a bead for REE separation comprise: (a) encapsulating one or more microbes in a PEGDA hydrogel to from microbe encapsulated particles; and (b) selecting microbe encapsulated particles having a length in at least one dimension between about 300 μm to about 500 μm; wherein the microbes are embedded within or on a surface of the particles.

In some embodiments, the microbes are embedded in a PEGDA hydrogel. In some embodiments, the microbes are embedded in the PEGDA by a free radical polymerization reaction to form a microbe encapsulated PEGDA gel. In some embodiments, the free radical polymerization reaction to form the microbe encapsulated PEGDA hydrogel includes (a) forming a precursor solution comprising a PEGDA monomer, a photoinitiator, and the microbes; (b) mechanically stirring the solution; and (c) polymerizing the solution with UV light. In some embodiments, the photoinitiator is 2,4,6-Trimethylbenzoylphenyl phosphonic acid ethyl ester (TPO-L).

In some embodiments, the microbes are added to the solution as a pellet. In some embodiments, the pellet comprises cells at a concentration of about 10 cells per milliliter (cells/mL), 10⁹ cells/mL, 10¹⁰ cells/mL, 10¹¹ cells/mL, 10¹² cells/mL, 10¹³ cells/mL, 10¹⁴ cells/mL, 10¹⁵ cells/mL, or any combination thereof, of the total volume of the bead. In some embodiments, the pellet comprises cells at a concentration between about 10⁸ cells/mL to 10¹⁵ cells/mL, about 10⁸ cells/mL to about 10¹¹ cells/mL, about 10⁹ cells/mL to about 10¹³ cells/mL, about 10¹⁰ cells/mL to about 10¹² cells/mL, about 10⁸ cells/mL to about 10¹³ cells/mL, about 10¹¹ cells/mL to about 10¹⁵ cells/mL, or about 10¹⁰ cells/mL to about 10¹⁵ cells/mL. In some embodiments, the pellet comprises cells at a concentration of about 10¹¹ cells/mL.

In some embodiments, the microbe encapsulated PEGDA hydrogel is crushed to form particles of various sizes. Crushing the microbe encapsulated gel can include pulverization and/or compression with force. The crushing step reduces the microbe encapsulated PEGDA gel to fine particles. In some embodiments, after crushing the microbe encapsulated PEGDA gel to form the crushed particles of various sizes, the crushed particles are passed through a sieve (e.g., a filter) that permits the separation of particles having an average size between about 150 μm to about 700 μm from the remainder of the particles. In some embodiments, the particles have average size in one and/or all dimensions of about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, 470 μm, about 480 μm, about 490 μm, about 500 μm, about 510 μm, about 520 μm, about 530 μm, about 540 μm, about 550 μm, about 560 μm, about 570 μm, about 580 μm, about 590 μm, about 600 μm, about 610 μm, about 620 μm, 630 μm, about 640 μm, about 650 μm, about 660 μm, about 670 μm, about 680 μm, about 690 μm, or about 700 μm. In some embodiments, the particles have an average size in one and/or all dimensions between about 150 μm to about 300 μm, about 300 μm to about 500 μm, or about 500 μm to about 700 μm. In some embodiments, the methods further comprise selecting particles having an average size of about 300 μm to about 500 μm.

In some embodiments, the particles have a regular and/or irregular shape. In some embodiments, the particles have an irregular cuboid, cube, sphere, ellipsoid, cone, triangular prism, cylindrical shape.

In some embodiments, the particle has a high cell density of microbes. It is contemplated that a high cell loading can act, at least in part, to enhance the saturation capacity of the biosorption material by increasing the number of available sites for REE binding. An increased number of REE binding ligands leads to a larger percentage of REE from the REE-containing material that complex with the REE microbes to form a REE-microbe complex (e.g., increased saturation capacity). In some embodiments, the increase in saturation capacity correlates with an increase in adsorption capacity (i.e., an increase in the number of Sc ions that complex with the microbes per unit volume or unit mass of the REE-containing material). It is contemplated that an increased saturation and adsorption capacity obviates the need from additional and energy exhaustive steps such as centrifugation and filtration in the process of separating REE from REE-containing material. In some embodiments, a high cell density does not correlate to increased absorption capacity. In some embodiments, the microbe is A. nicotianae.

In some embodiments, the high cell density of the microbes is about 10⁸ cells/mL, 10⁹ cells/mL, 10¹⁰ cells/mL, 10¹¹ cells/mL, 10¹² cells/mL, 10¹³ cells/mL, 10¹⁴ cells/mL, 10¹⁵ cells/mL, or any combination thereof, of the total volume of the bead. In some embodiments, the bead for REE separation has a high cell density between about 10⁸ cells/mL to 10¹⁵ cells/mL, about 10⁸ cells/mL to about 10¹¹ cells/mL, about 10¹² cells/mL to about 10¹³ cells/mL, about 10¹⁰ cells/mL to about 10¹² cells/mL, about 10⁸ cells/mL to about 10¹³ cells/mL, about 10¹¹ cells/mL to about 10¹⁵ cells/mL, or about 10¹⁰ cells/mL to about 10¹⁵ cells/mL.

In some embodiments, the particles have a high cell density of microbes, where the cell density is about 0.2 to about 4 g of cells/mL, about 0.2 cells/mL, about 0.3 cells/mL, about 0.4 cells/mL, about 0.5 cells/mL, about 0.6 cells/mL, about 0.7 cells/mL, about 0.8 cells/mL, about 0.9 cells/mL, about 1 cells/mL, about 1.1 cells/mL, about 1.2 cells/mL, about 1.3 cells/mL, about 1.4 cells/mL, about 1.5 cells/mL, about 1.6 cells/mL, about 1.7 cells/mL, about 1.8 cells/mL, about 1.9 cells/mL, about 2 cells/mL, about 2.1 cells/mL, about 2.2 cells/mL, about 2.3 cells/mL, about 2.4 cells/mL, about 2.5 cells/mL, about 2.6 cells/mL, about 2.7 cells/mL, about 2.8 cells/mL, about 2.9 cells/mL, about 3 cells/mL, about 3.1 cells/mL, about 3.2 cells/mL, about 3.3 cells/mL, about 3.4 cells/mL, about 3.5 cells/mL, about 3.6 cells/mL, about 3.7 cells/mL, about 3.8 cells/mL, about 3.9 cells/mL, about 4 cells/mL, or any combination thereof, of the total volume of the particle. In some embodiments, the particle for Sc separation has a high cell density between about 0.5 cells/mL to 2 cells/mL, about 0.2 cells/mL to about 4 cells/mL, about 0.5 cells/mL to about 3 cells/mL, about 2 cells/mL to about 4 cells/mL, about 1 cells/mL to about 2 cells/mL, about 1.5 cells/mL to about 2 cells/mL, or about 1 cells/mL to about 3 cells/mL. In some embodiments, the microbes are A. nicotianae microbes.

In some embodiments, the high cell density of the microbes is at least about 10 weight percent (wt %), 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, or more of the total weight of the bead or at least about 20 volume percent (vol %), at least about 25 vol %, at least about 30 vol %, at least about 35 vol %, at least about 40 vol %, at least about 45 vol %, at least about 50 vol %, at least about 55 vol %, at least about 60 vol %, at least about 65 vol %, at least about 70 vol %, at least about 75 vol %, at least about 80 vol %, at least about 85 vol %, at least about 90 vol %, at least about 95 vol % or more of the total volume of the particle. In some embodiments, the microbes are A. nicotianae microbes.

In some embodiments, the particles have a cell density of about 1 g/mL. A cell density of about 1 g/mL can provide an optimal balance between cell loading and absorption capacity. For example, a higher REE absorption capacity can be achieved in a particle having lower a cell density of about 1 g/mL as compared to particles having a higher cell density of 2 g/mL. In some embodiments, particles having a cell density of 1 g/mL achieve greater absorption capacity as compared to particles having a cell density of about 2 g/mL, about 2.5 g/mL, about 3 g/mL, about 3.5 g/mL, or about 4 g/mL.

In some embodiments, the high adsorption capacity of the microbes is at least about 1 milligram (mg), at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 34 mg, at least about 35 mg, at least about 36 mg, at least about 37 mg, at least about 38 mg, at least about 39 mg, at least about 40 mg, at least about 41 mg, at least about 42 mg, at least about 43 mg, at least about 44 mg, at least about 45 mg, at least about 46 mg, at least about 47 mg, at least about 48 mg, at least about 49 mg, or at least about 50 mg of Sc per gram (g) of dried particle. In some embodiments, the microbes are A. nicotianae microbes.

In some embodiments, the high adsorption capacity of the microbes is at least about 30 mg, at least about 35 mg, at least about 40 mg, at least about 45 mg, at least about 50 mg, at least about 55 mg, at least about 60 mg, at least about 70 mg, at least about 75 mg, at least about 80 mg, at least about 90 mg, at least about 100 mg of Sc g of microbe. In some embodiments, the microbes are A. nicotianae microbes.

In another embodiment, the microbes are encapsulated within and/or on a surface of the particles. When the microbes are encapsulated within and/or on the surface of the bead, the particles are able to efficiently bind the REEs by increasing the accessibility of the microbes for binding. Once the REE-containing material is flowed on and/or through the particle, the microbes are able to capture the REEs both within and on the surface of the bead, which optimizes the adsorption capacity of the bead by increasing the ratio of available binding sites (i.e., microbes) to total volume of the particle.

In some embodiments, the particles are porous. The porous particles enable the flow of the REE containing material to contact not only the exterior surface, but also, the interior surface of the particle thereby increase the saturation and absorption capacity of the particle for REEs (i.e., increased accessibility). The pore size is optimized to facilitate the diffusion of REEs into and out of the particle (e.g., the pore size is large enough to enable a flow of REEs into and out of the particle without trapping the REE within the matrix of the particle). The pore size also prevents microbes cocci having an average diameter of at least about 1 μM from diffusing into and out of the particle thereby enabling a high cell loading of the microbe in the particle. In some embodiments, the particles have an average pore diameter of about 40 nm to about 250 nm. For example, the particles have a pore diameter of at least about 40 nm, at least about 45 nm, at least about 50 nm, at least about 55 nm, at least about 65 nm, at least about 70 nm, at least about 75 nm, at least about 80 nm, at least about 85 nm, at least about 90 nm, at least about 95 nm, at least about 100 nm, at least about 105 nm, at least about 110 nm, at least about 115 nm, at least about 120 nm, at least about 125 nm, at least about 130 nm, at least about 135 nm, at least about 140 nm, at least about 145 nm, at least about 150 nm, at least about 150 nm, at least about 160 nm, at least about 165 nm, at least about 170 nm, about least about 175 nm, at least about 180 nm, at least about 185 nm, at least about 190 nm, at least about 195 nm, at least about 200 nm, at least about 205 nm, at least about 210 nm, at least about 215 nm, at least about 220 nm, at least about 225 nm, at least about 230 nm, at least about 235 nm, at least about 240 nm, at least about 245 nm, at least about 250 nm. In some embodiments, the particle has a pore diameter between about 50 nm to about 200 nm, about 50 nm to about 100 nm, about 40 nm to about 100 n, about 60 nm to about 190 nm, about 70 nm to about 100 nm, about 80 nm to about 200 nm, about 80 nm to about 180 nm, about 60 nm to about 150 nm, or 70 nm to 200 nm.

In some embodiments, the pores are evenly distributed throughout the particles. The evenly distributed pores are attributable to the entrapment, rather than chemical cross-linking of the microbes of the SNPs. In some embodiments, the microbes are homogenously distributed throughout the microbe particle.

In some embodiments, the methods further comprise incorporating the particle into a column, membrane, bead, or combination thereof.

In some embodiments, the biosorption/adsorption media are related to Sc separation; however, biosorption/adsorption media can be made similarly applicable to any REE including La, Ce, Pr, Nd, Pm, Sm, Eu, Gd, Tb, Dy, Ho, Er, Tm, Yb, Lu, Sc, and/or Y. For example, in some embodiments, the biosorption/adsorption media can encapsulate microbes capable of facilitating the separation of one or more REEs. The embodiments of the biosorption/adsorption media are not limited to Sc separation.

### Methods

Also provided herein are methods of preferentially separating REEs, including, but not limited to, Sc from REE-containing materials using microbes. These methods further comprise separating total REEs and/or groups of one or more REEs from the REE-containing materials.

In one aspect provided herein are methods for preferentially separating Sc from a REE containing material comprising the steps of: (a) contacting microbes with the REE containing material at a pH between about 3 to about 4 to form Sc-microbe complexes; and (b) separating the Sc from the microbes by contacting the Sc-microbe complexes with a solution comprising an organic chelator, wherein the microbe is A. nicotianae microbes. In some embodiments, the steps described are executed once. In other embodiments, the steps or a portion of the steps are executed more than once, for example, 2, 3, 4, 5, 6, 7, 8, 9, 10 or more times. In some embodiments, the steps or portions of the steps are executed more than once with more than one REE-containing material, for example with 1, 2, 3, 4, 5, 6, 7, 8, 9, 10 or more REE-containing materials.

In another aspect provided herein are methods for preferentially separating Sc and total REEs from a REE containing material comprising the steps of: (a) contacting microbes embedded within a first solid support with the REE containing material at a pH of about 3 to about 4 to form Sc-microbe complexes; (b) collecting the REE containing material, wherein the REE material contains substantially no Sc after contact with the microbes embedded within the first solid support; and (c) contacting microbes embedded within a second solid support with REE material containing substantially no Sc to form REE-microbe complexes. In other embodiments, the steps or a portion of the steps are executed more than once, for example, 2, 3, 4, 5, 6, 7, 8, 9, 10 or more times. In some embodiments, the steps or portions of the steps are executed more than once with more than one REE-containing material, for example with 1, 2, 3, 4, 5, 6, 7, 8, 9, 10 or more REE-containing materials.

In some embodiments, the steps or portions of the steps are repeated until at least about 100%, at least about 90%, at least about 80%, at least about 70%, at least about 60%, at least about 50%, at least about 40%, at least about 30%, at least about 20%, or at least about 10% of the Sc and/or other REEs are separated from the REE containing material.

In some embodiments, the REE containing material is pre-processed prior to contacting with the microbes to adjust the pH of the REE containing material. In some embodiments, the REE containing material is adjusted to a pH of about 3 to 4 prior to contacting the REE containing material to the microbes. Lanthanides and yttrium can be selectively extracted from REE containing materials at a pH between 5-6; however, Sc has low solubility in the pH range of 5-6, precluding the separation of Sc from REE containing material. However, Sc is soluble at a pH of about 3-4 and a high selectively for A. nicotianae microbes, enabling the separation of Sc from other REEs and REE containing material upon contact with A. nicotianae microbes at a pH between about 3-4. In some embodiments, the pH of the REE containing material is incrementally adjusted from a pH of about 3 to about 4 upon contact with A. nicotianae microbes. In some embodiments, the pH of the REE containing material is incrementally adjusted from 3 to 3.4, 3.4 to 3.6, and 3.6 to 3.8 upon contact with A. nicotianae microbes.

In some embodiments, the REE containing material is pre-processed prior to contacting with the microbes to reduce Fe(III) in the REE containing material to Fe(II). In some embodiments, reducing Fe(III) to Fe(II) can prevent co-elution of Sc with Fe(III) such that Sc and Fe can be preferentially separated. Fe(II), unlike Fe (III), will not absorb to the microbes, whereas Sc will adsorb to the microbes thereby allowing for the separation of Sc from Fe in the REE containing material.

In some embodiments, the Sc is separated from the Sc-microbe complexes with a solution comprising an organic chelator. In some embodiments, the organic chelator has a low molecular weight. For example, a low molecular weight of about 50 g/mol, 60 g/mol, 70 g/mol, 80 g/mol, 90 g/mol, 100 g/mol, 110 g/mol, 120 g/mol, 130 g/mol, 140 g/mol, 150 g/mol, 160 g/mol, 170 g/mol. 180 g/mol, 190 g/mol, about 200 g/mol, 210 g/mol, 220 g/mol, 230 g/mol, 240 g/mol. 250 g/mol, 260 g/mol, 270 g/mol, 280 g/mol, 290 g/mol, or 300 g/mol. In some embodiments the organic chelator molecular is selected from the group consisting of citrate, ethylenediamine, and ethylenediaminetetraacetic acid (EDTA). In some embodiments, the citrate organic chelator is selected from the group consisting of sodium citrate, magnesium citrate, potassium citrate, calcium citrate, trisodium citrate dihydrate, and butetamate citrate. While Sc can be desorbed from the Sc-microbe complex using a low pH (e.g., <1), to prevent harsh treatment, which is problematic for column stability, the solution can be adjusted to have a pH between about 5 to about 6. By introducing a solution having a pH between about 5 to about 6, the Sc is desorbed from the microbes and precipitated. This enables the isolation of Sc with no or substantially no contamination from other REE and/or non-REEs. In some embodiments, the Sc is separated from the Sc-microbe complexes with a solution comprising an organic chelator having a pH between about 5 to about 6. In some embodiments, the Sc is separated from the Sc-microbe complexes with a solution comprising citrate having a pH between about 5 to about 6. In some embodiments, the concentration of the organic chelator in the solution is between about 10 mM to about 100 mM. In some embodiments, the concentration of the organic chelator in the solution is about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. In some embodiments, the microbes are embedded within a polymeric particle. In some embodiments, the organic chelator to microbe ratio is about 1:40. In some embodiments, the organic chelator is citrate.

In some embodiments, the Sc is preferentially separated from REEs other than Sc. In some embodiments, Sc is preferentially separated from one or more of La, Ce, Pr, Nd, Pm, Sm, Eu, Gd, Tb, Dy, Ho, Er, Tm, Yb, Lu, and/or Y.

In some embodiments, Sc is preferentially separated from non-REEs. In some embodiments, Sc is preferentially separated from one or more non-REE metals and/or radionucleotides. In some embodiments, the non-REE metals are one or more of Fe, Ca, Al, Mg, Zn, Ni, Li, K, Mn, Cu, and/or Na. In some embodiments, the one or more radionucleotides are uranyl (U) and/or thorium (Th). In some embodiments, REEs other than Sc are preferentially separated from non-REEs.

In some embodiments, the methods further comprise separating REEs other than Sc from REE containing material. In some embodiments, the methods further comprise separating Sc and then separating the remainder of the REEs from the same REE containing material. Accordingly, the methods include separation of total REEs (i.e., Sc and REEs other than Sc) from REE containing material. In some embodiments, the methods comprise a two-step process of selectively absorbing and desorbing Sc from the microbes and then reintroducing the REE containing material containing no or substantially no Sc to the microbes to selective absorb and desorb REEs other than Sc.

In some embodiments, the methods of preferentially separating Sc and total REEs from REE containing material comprises a step of contacting A. nicotianae microbes with REE containing material pre-processed to have a pH of about 3 to about 4 to from a Sc-microbe complex, wherein the microbes are embedded within a first solid support. In some embodiments, the methods further comprise separating Sc from the microbes by contacting the Sc-microbe complex with a solution having an organic chelator. In some embodiments, the organic chelator is citrate and the solution has a pH of about 5 to about 6.

In some embodiments, the methods of preferentially separating Sc and total REEs from REE containing material further comprise collecting the filtrate (i.e., REE containing material after the containing step that forms Sc-microbe complexes). In some embodiments, the methods further comprise contacting microbes embedded within a second solid support with the filtrate to from REE-microbe complexes. In some embodiments, the methods comprise separating the total REEs from the microbes by contacting REE-microbe complexes with a solution having a pH of about 5 to about 6. In some embodiments, the solution comprises an organic chelator such as citrate. In another embodiment, the methods comprise separating the total REEs from the microbes by contacting REE-microbe complexes with a solution comprising a strong acid. Non-limiting examples of strong acids include phosphoric acid (H₃PO₄), hydrogen chloride (HCl), nitric acid (HNO₃), or sulfuric acid (H₂SO₄). In some embodiments, the solution comprising the strong acid has a pH less than about 5, about 4, about 3, about 2, or about 1.

In some embodiments, the microbes embedded within the second solid support are genetically engineered microbes for use in separating REEs from non-REEs. In some embodiments, the microbes are genetically engineered to express REE binding ligands, such as lanthanide binding tags (LBT). Suitable microbes, including suitable genetically modified microbes are descripted in US Publication No. 2018/0195147, which is incorporated by reference in its entirety.

In some embodiments, the methods of preferentially separating Sc and total REEs from REE containing material further comprise adjusting the pH of the filtrate to about 5 to precipitate and filter out non-REE components from the REE containing material. In some embodiments, the non-REE components are Fe, Al, or both. In some embodiments, the precipitated non-REEs are filtered from the precipitate prior to contacting the filtrate with the microbes embedded within as second solid support.

In some embodiments, the Sc is separated with a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE and/or non-REE.

In some embodiments, REEs other than Sc are separated with a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to Sc and/or non-REE.

In some embodiments, the Sc is separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other element.

In some embodiments, REEs other than Sc are separated with a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other element.

In some embodiments, the Sc is separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to radionucleotide.

In some embodiments, REEs other than Sc are separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to radionucleotide.

In some embodiments, the Sc is separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to La, Ce, Pr, Nd, Pm, Sm, Eu, Gd, Tb, Dy, Ho, Er, Tm, Yb, Lu, and/or Y.

In some embodiments, the Sc is separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to Fe, Ca, Al, Mg, Zn, Ni, Mg, and/or Na.

In some embodiments, REEs other than Sc are separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to Fe, Ca, Al, Mg, Zn, Ni, Mg, and/or Na.

In some embodiments, the Sc is separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to U and h.

In some embodiments, REEs other than Sc separated with a purity at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to U and Th.

In some embodiments, the microbes are added to a column prior to contacting the microbes encoding at least one REE binding ligand with the REE containing material. In some embodiments, prior to adding the microbes to the column, the microbes are formulated within or to the surface of a solid structure (e.g., a bead, capsule, and/or particle). When added to the column, the microbes are used, as defined conventionally in column chromatography, as the stationary phase. This enables a continuous flow system in which REE containing material is introduced to the column and flows from the top to the bottom of the column.

In some embodiments, the present disclosure provides methods for preferentially separating Sc in a single step. Single step separation occurs when the REE-containing material is introduced to the microbes and results in the isolation and purification of Sc with no or substantially no other element.

In some embodiments, the present disclosure provides methods for preferentially separating Sc and total REEs in two steps. Two step separation occurs when the REE-containing material is introduced to the microbes and results in the isolation and purification of Sc with no or substantially no other element and then reintroducing the REE-containing material to microbes resulting in the isolation and purification and REEs other than Sc.

In some embodiments, the methods for preferentially separating Sc is continuous and uninterrupted by additional energy-intensive steps such as centrifugation and/or filtration. In other embodiments, the methods for preferentially separating Sc comprise an additional step of centrifugation filtration, or both.

In some embodiments, the methods for preferentially separating Sc and/or total REEs is continuous and uninterrupted by additional energy-intensive steps such as centrifugation and/or filtration. In other embodiments, the methods for preferentially separating Sc and/or total REEs comprise an additional step of centrifugation filtration, or both.

In some embodiments, A. nicotianae microbes selectively bind to Sc due to the smaller ionic character of Sc relative to other REEs or non-REEs.

The REE-containing material may be any material known to contain or suspected to contain REE. In some embodiments the material is a solid material, a semi-solid material, or an aqueous medium. In a preferred embodiment, the material is an aqueous solution. Non-limiting examples of suitable materials for use in extraction of REE include rare earth ores (e.g., bastnaesite, monazite, loparite, and the lateritic ion-adsorption clays), geothermal brines, coal, coal byproducts, mine tailings, phosphogypsum, electronic waste, bauxite, acid leachate of solid source materials, REE solution extracted from solid materials through ion-exchange methods, or other ore materials, such as REE-containing clays, volcanic ash, organic materials, and any solids/liquids that react with igneous and sedimentary rocks.

In some embodiments, the REE-containing material is a low-grade material wherein the REEs are present in less than about 2 wt % of the total weight of the low-grade material. In other embodiments, the REE-containing material is a high-grade material, wherein the REE are present in greater than about 2 wt % of the total weight of the high-grade material.

In some embodiments, the REE-containing material comprises less than about 5 wt %, less than about 10 wt %, less than about 15 wt %, less than about 20 wt %, less than about 25 wt %, less than about 30 wt %, less than about 35 wt %, less than about 40 wt %, less than about 45 wt %, less than about 50 wt % Sc and/or total REEs of the total weight of the REE-containing material.

In some embodiments, the REE containing material comprises a substantially greater concentration of non-REE metals relative to Sc. In some embodiments, the REE containing material comprises substantially more Fe and/or Al relative to Sc. In some embodiments, the concentration of the non-REE metals relative to Sc is 2 times to 10,000 times greater than the concentration of Sc. In some embodiments, the concentration of the non-REE metals relative to Sc is 2 times, 10 times, 100 times, 200 times, 300 times, 400 times, 500 times, 600 times, 900 times, 1,000 times, 2,000 times, 3,000 times, 4,000 times, 5,000 times, 6,000 times, 7,000 times, 8,000 times, 9,000 times, or 10,000 times greater than the concentration of Sc. In some embodiments, the concentration of the non-REEs are present in the REE containing material in a concentration three, four, five, or more orders of magnitude higher than the concentration of Sc.

The microbes can also be used for recovering REE from recycled REE-containing products such as, compact fluorescent light bulbs, electroceramics, fuel cell electrodes, NiMH batteries, permanent magnets, catalytic converters, camera and telescope lenses, carbon lighting applications, computer hard drives, wind turbines, hybrid cars, x-ray and magnetic image systems, television screens, computer screens, fluid cracking catalysts, phosphor-powder from recycled lamps, and the like. These materials are characterized as containing amounts of REE, including, for example, La, Ce, Pr, Nd, Pm, Sm, Eu, Gd, Tb, Dy, Ho, Er, Tm, Yb, Lu, and/or Y.

In some embodiments, the material is pre-processed prior to providing the microbes. Non-limiting examples of suitable pre-processing includes acid leaching, bioleaching, ion-exchange extraction, pH adjustment, iron oxide precipitation, temperature cooling (e.g., geothermal brines). In other embodiments, prior to providing the microbes, the REE-containing material is refined to remove at least a portion of non-REE metals. In some embodiments, the non-REE metals are extracted using microbes, for example, A. nicotianae microbes.

In some embodiments, at least a portion of the microbes are attached (i.e., immobilized) to a surface of a solid support prior to contacting with a REE-containing material. It is contemplated that microbe immobilization in biosorption medium for use in flow-through setups allows for complete (or substantially complete) separation of Sc from REE-containing mixed metal solutions in a single step. In one embodiment, about 20%, about 30%, about 40%, about 50%, about 60%, about 70%, about 80%, about 90%, about 91%, about 95%, about 97%, about 98%, about 99%, or 100% of Sc in the REE-containing material (e.g., mixed metal solution) is extracted in a single step. In some embodiments, about 1%, 5%, 10%, 15%, 20%, about 30%, about 40%, about 50%, about 60%, about 70%, about 80%, about 90%, about 91%, about 95%, about 97%, about 98%, about 99%, or 100% of the Sc in the REE-containing material (e.g., mixed metal solution) is extracted in a single step as compared to an amount of REE extracted in a single step using conventional extraction methods.

The binding of Sc to the microbes can be reversible. In some embodiments, at least a portion of the Sc in the microbe-REE complex is desorbed (i.e., removed or separated) from the microbes. In another preferred embodiment, wherein the removal step is performed using an amount of citrate.

The microbes can also be reused. In some embodiments, the methods further comprise removing the Sc and/or REE from the microbes to regenerate microbes. The microbes can be used 2, 3, 4, 5, 6, 7, 8, 9, 10, 15, 20, 25, 30, or more times. In other embodiments, the microbes are single use. The microbes can be re-conditioned by any means known to one of skill in the art. For example, the microbes may be cleaned with deionized (DI) water, a dilute saline solution, and/or a buffer solution to wash off the citrate to re-generate microbes. In one embodiment, the methods further comprise reusing the regenerated microbes to carry out the extraction of REE from REE-containing material.

The microbes can be reused 2, 3, 4, 5, 6, 7, 8, 9, 10, 15, 20, 25, 30 or more times while also maintaining their high adsorption capacity. In some embodiments, the microbes maintain an adsorption capacity of about 1.0 mg of Sc and/or total REE, about 1.5 mg of Sc and/or total REE, about 2.0 mg of Sc and/or total REE, about 2.5 mg of Sc and/or total REE per g of the particle during each of the adsorption cycles.

Also provided herein are methods of preferentially separating one or more REEs, from REE-containing materials using microbes that allows for increased scalability and industrially relevant flow rates. In some embodiments, the one or more REEs is Sc.

In one aspect provided herein are methods for preferentially separating one or more REEs from a REE containing material comprising the steps of: (a) contacting microbes with the REE containing material at a pH between about 3 to about 4 to form REE-microbe complexes; wherein the microbes are encapsulated in a PEGDA hydrogel and (b) separating the one or more REEs from the microbes by contacting the REE-microbe complexes with a solution comprising an organic chelator. In some embodiments, the steps described are executed once. In other embodiments, the steps or a portion of the steps are executed more than once, for example, 2, 3, 4, 5, 6, 7, 8, 9, 10 or more times. In some embodiments, the steps or portions of the steps are executed more than once with more than one REE-containing material, for example with 1, 2, 3, 4, 5, 6, 7, 8, 9, 10 or more REE-containing materials. In some embodiments, the one or more REEs is Sc.

In another aspect provided herein are methods for preferentially separating one or more REEs from an REE containing material comprising the steps of: (a) adding microbes embedded within PEGDA hydrogel to a column; (b) introducing to the microbes embedded within PEGDA hydrogel the REE containing material at a flow rate of about 2×10⁻³ m/s to 4×10⁻³ meters per second (m/s) and at a pH of about 3 to about 4 to form REE-microbe complexes; (c) separating the REEs from the microbes by contacting the REE-microbe complexes with a solution comprising an organic chelator. In other embodiments, the steps or a portion of the steps are executed more than once, for example, 2, 3, 4, 5, 6, 7, 8, 9, 10 or more times. In some embodiments, the steps or portions of the steps are executed more than once with more than one REE-containing material, for example with 1, 2, 3, 4, 5, 6, 7, 8, 9, 10 or more REE-containing materials. In some embodiments, the one or more REEs is Sc.

In some embodiments, the column is an industrially length column having a diameter of about 0.1 meters to about 2 meters and a length of about 0.3 meters to about 10 meters. For example, a diameter of about 0.1 meters, about 0.2 meters, about 0.3 meters, about 0.4 meters, about 0.5 meters, about 0.6 meters, about 0.7 meters, about 0.8 meters, about 0.9 meters, about 1 meters, about 1.1 meters, about 1.2 meters, about 1.3 meters, about 1.4 meters, about 1.5 meters, about 1.6 meters, about 1.7 meters, about 1.8 meters, about 1.9 meters, or about 2 meters. For example, a length of about 0.3 meters, about 0.5 meters, about 1 meter, about 1.5 meters, about 2 meters, about 2.5 meters, about 3 meters, about 3.5 meters, about 4 meters, about 4.5 meters, about 5 meters, about 5.5 meters, about 6 meters, about 6.5 meters, about 7 meters, about 7.5 meters, about 8 meters, about 8.5 meters, about 9 meters, about 9.5 meters, or about 10 meters.

In some embodiments, the REE containing material is introduced to the column at an industrially relevant flow rate, allowing for the efficient and commercially relevant separation of REEs and/or groups of REEs from REE containing material. In some embodiments, the REE containing material is introduced to the column at a flow rate of 0.5×10⁻³ m/s to 4×10⁻³ m/s. In some embodiments, the flow rate is about 0.5×10⁻³ m/s, about 0.6×10⁻³ m/s, about 0.7×10⁻³ m/s, about 0.8×10⁻³ m/s, 0.9×10⁻³ m/s, about 1×10⁻³ m/s, about 1.1×10⁻³ m/s, about 1.1×10⁻³ m/s, 1.2×10⁻³ m/s, about 1.3×10⁻³ m/s, about 1.4×10⁻³ m/s, about 1.5×10⁻³ m/s, about 1.6×10⁻³ m/s, about 1.7×10⁻³ m/s, about 1.8×10⁻³ m/s, about 1.9×10⁻³ m/s, about 2×10⁻³ m/s, about 2.1×10⁻³ m/s, about 2.2×10⁻³ m/s, about 2.3×10⁻³ m/s, about 2.4×10⁻³ m/s, about 2.5×10⁻³ m/s, about 2.6×10⁻³ m/s, about 2.7×10⁻³ m/s, about 2.8×10⁻³ m/s, about 2.9×10⁻³ m/s, about 3.0×10⁻³ m/s, about 3.1×10⁻³ m/s, about 3.2×10⁻³ m/s, about 3.3×10⁻³ m/s, about 3.4×10⁻³ m/s, about 3.5×10⁻³ m/s, about 3.6×10⁻³ m/s, about 3.7×10⁻³ m/s, about 3.8×10⁻³ m/s, about 3.9×10⁻³ m/s, or about 4.9×10⁻³ m/s.

In some embodiments, the one or more REEs are introduced into the column at an industrially relevant concentration, allowing for the efficient and commercially relevant separation of REEs from REE containing material. An industrially relevant concentration can include concentrations of REEs without dilution of the REE containing material. In some embodiments, the methods include separating REEs from REEs containing materials such as geothermal brines having a concentration of REEs as low as 1 μM. In some embodiments, the REE containing material comprises the one or more REEs at a concentration of about 1 μM to about 3.0 mM. In some embodiment, REE containing material comprises the one or more REEs at a concentration of about 1 μM, about 5 μM, about 10 μM, about 50 μM, about 100 μM, about 200 μM, about 300 μM, about 400 μM, about 500 μM, about 600 μM, about 700 μM, about 800 μM, about 900 μM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, or about 3.0 mM. In some embodiments, the one or more REEs is Sc and is introduced to the column at a concentration of about 2.0 mM.

In some embodiments, the one or more REEs are separated (e.g., desorbed) from the microbes by contacting the REE-microbe complexes with a solution comprising an organic chelator. In some embodiments, the one or more REEs is separated from the REE-microbe complexes with a solution comprising having a pH between about 5 to about 6. In some embodiments, the concentration of the organic chelator in the solution is between about 10 mM to about 100 mM. In some embodiments, the concentration of the organic chelator in the solution is about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. In some embodiments, the organic chelator to microbe ratio is about 1:40. In some embodiments, the organic chelator is citrate. In some embodiments, the organic chelator is citrate and the citrate is present at a concentration of about 50 mM.

In some embodiments, a concentration of the one or more REEs after desorption from the REE-microbe complexes is greater than a concentration of the one or more REEs in the REE containing material. The microbes are capable of adsorbing a greater number of the one or more REEs than is initially introduced to the column, allowing for a large-scale separation of one or more REEs by continuous introduction and/or flow of the REE containing material over the encapsulated microbes. In some embodiments, a concentration of the one or more REEs desorbed from the REE-microbe complexes is about 20 mM to about 40 mM. In some embodiments, a concentration of the one or more REEs desorbed from the REE-microbe complexes is about 20 mM, about 22 mM, about 24 mM, about 26 mM, about 28 mM, about 30 mM, about 32 mM, about 34 mM, about 36 mM, about 38 mM, or about 40 mM. In some embodiments, a concentration of the one or more REEs desorbed from the REE-microbe complexes is at least about 5 times greater than an initial concentration of the one or more REEs in the REE containing material. For example, at least about 5 times, at least about 10 times, at least about 15 times, at least about 20 times, at least about 25 times, or at least about 30 times greater than an initial concentration of the one or more REEs in the REE containing material. In some embodiments, the one or more REEs is Sc, which is introduced to the column at a concentration of about 2 mM, and the concentration of Sc desorbed from the column is about 32 mM. In some embodiments, the one or more REEs is Sc, and the concentration of Sc desorbed at a concentration that is 15 times greater than the initial concentration of Sc.

In some embodiments, the one or more REEs and/or groups of one or more REEs are separated in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE and/or group of REEs.

In some embodiments, the one or more REEs and/or groups of one or more REEs are separated in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other element.

In some embodiments, the one or more REEs and/or groups of one or more REEs are separated in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any non-REE component.

In some embodiments, the methods relate to Sc separation; however, the methods can be made similarly applicable to the separation of any REE including La, Ce, Pr, Nd, Pm, Sm, Eu, Gd, Tb, Dy, Ho, Er, Tm, Yb, Lu, Sc, and/or Y. For example, in some embodiments, the methods facilitate the separation of one or more REEs. The methods provided herein are not limited to Sc separation.

Aspects of the disclosure provide a kit of parts comprising: (a) A. nicotianae microbes (b) a solution comprising an organic chelator; and (c) instructions for differentially separating Sc from a REE-containing material. In some embodiments, the kit of parts further comprises genetically engineered microbes comprising an exogenous nucleic acid sequence encoding at least one REE binding ligand for separating REEs other than Sc from non-REEs in REE-containing material.

Aspects of the disclosure provide a kit of parts comprising: (a) genetically engineered microbes comprising an exogenous nucleic acid sequence encoding at least one REE binding ligand encapsulated in a PEGDA; and (b) instructions for differentially separating one or more REEs from a REE-containing material. In some embodiments, the one or more REEs is Sc.

### EXAMPLES

### Example 1: Bio-Based Material for Rare Earth Element Separation

Previous findings suggest that lanthanides and yttrium can be extracted with high selectivity from a number of feedstock leachates in the pH 5-6 range, including coal products, geothermal brines, mine tailings, ores, and electronic waste [1-4]. However, a significant decrease in REE purity is observed at lower pH (e.g., pH 4) as a consequence of elevated Al concentrations, precluding a lower pH extraction step [4]. As such the low solubility of Sc in the pH 5-6 range precludes a single-step biosorption/desorption process for recovery of lanthanides and Sc.

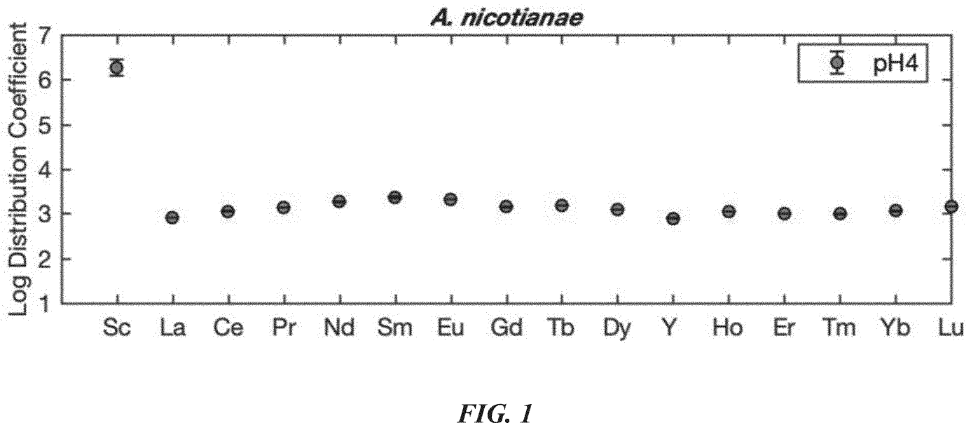

Accordingly, Sc was tested for selective extraction at pH 4. Biosorption experiments with a synthetic solution containing equimolar concentrations of Ln³⁺, Y, and Sc, and lacking non-REE competitors (i.e., the innate selectivity) revealed the strong preference of A. nicotianae for Sc over lanthanides at pH 4 (FIG. 1). While the K_{d} values for lanthanides and Y differed by less than an order of magnitude, the K_{d} value for Sc was ˜three orders of magnitude higher than for Sm, the lanthanide with the highest affinity for the cell surface. Based on these data, a method for the selective recovery of Sc from coal feedstock leachates was pursued.

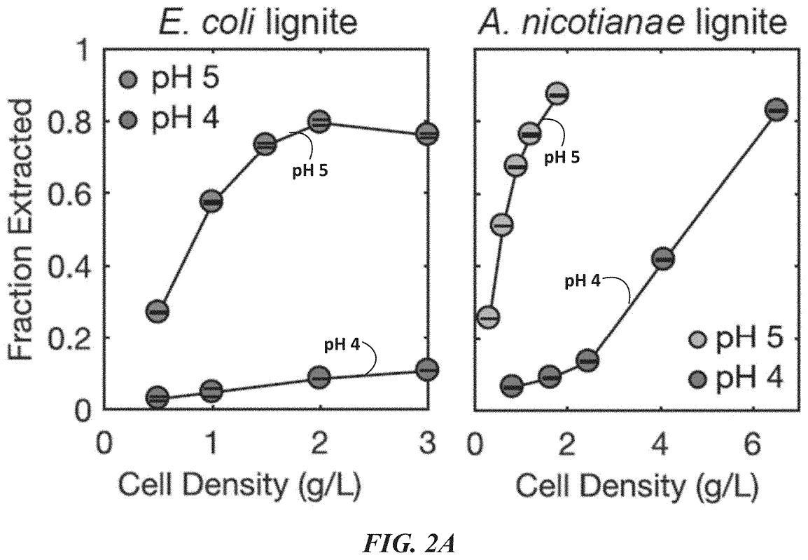

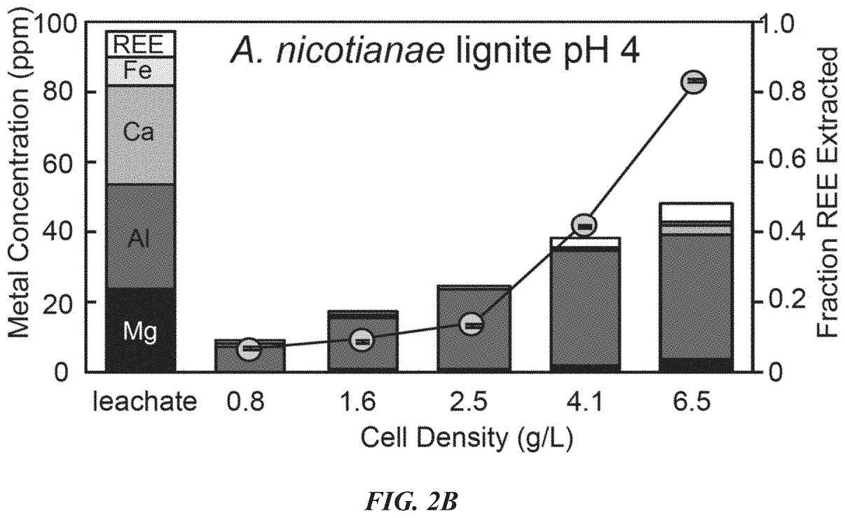

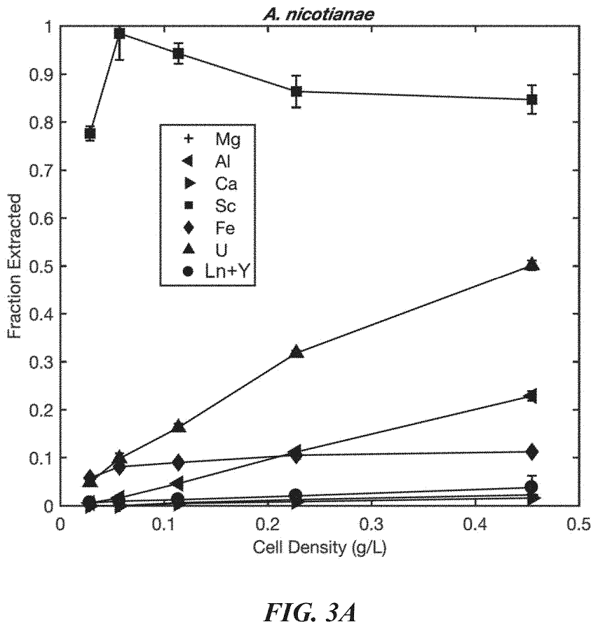

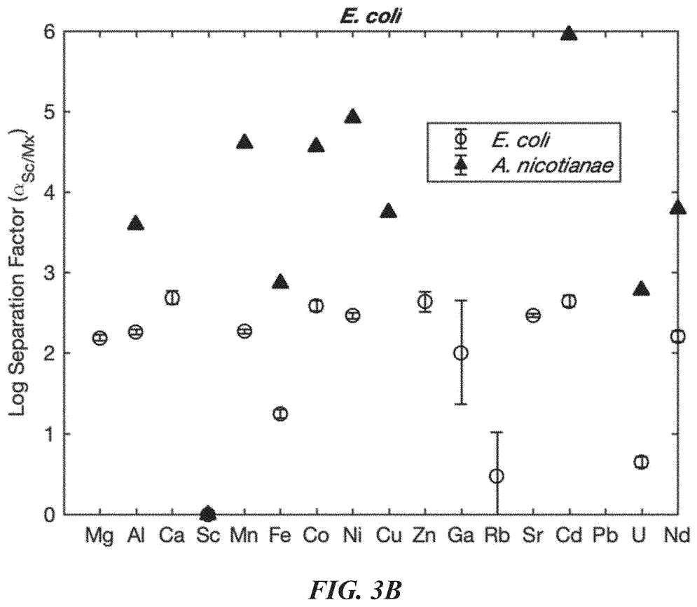

In contrast to Ln³⁺ and Y (FIG. 2A-2B), Sc can be extracted with high efficiency at pH 3-4 by A. nicotianae in lignite leachate (FIG. 3A). Notably, Sc was not extracted in a mock biosorption assay lacking cells, suggesting that Sc extraction is cell mediated and not a product of abiotic precipitation. In contrast to Sc, the extraction efficiencies of competitive metals (Al, U. Mg, Ca, Fe) decreased as a function of cell density (FIG. 3A). The extraction efficiency of Ln³⁺ and Y was negligible throughout this range (i.e., less than 1% extracted at the lowest cell density). Determination of the separation factor for Sc relative to each metal revealed αSc,Mx values at or greater than 3000 for all metals, including Nd, highlighting the general selectivity of A. nicotianae for Sc (FIG. 3B).

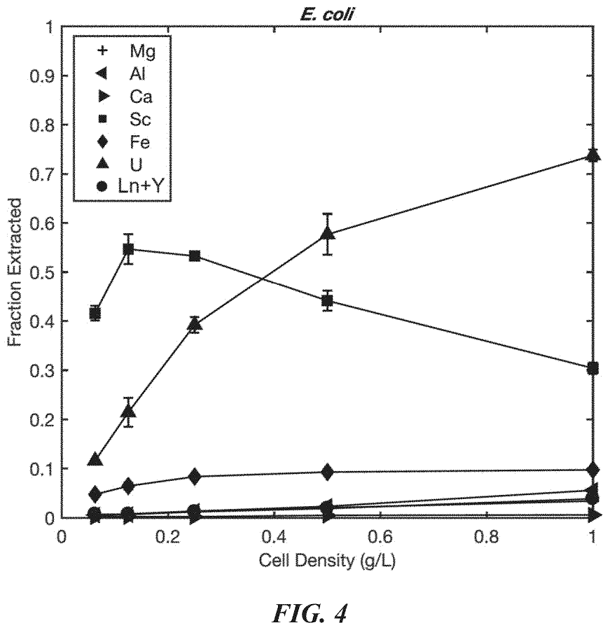

Biosorption assays over a similar range of cell densities of E. coli with lanthanide binding tags displayed on the cell surface yielded qualitatively similar trends for Sc and non-REEs, but with only 50% Sc extraction and lower selectivity relative to non-REEs (FIG. 4). Thus, the high cell surface affinity for Sc enables selective Sc recovery at pH 4 with low biomass concentrations. This suggests that the high cell surface affinity for Sc enables selective Sc recovery at low pH with low biomass concentrations.

### Fabrication and Characterization of Microbe Encapsulated SiO₂ Gel

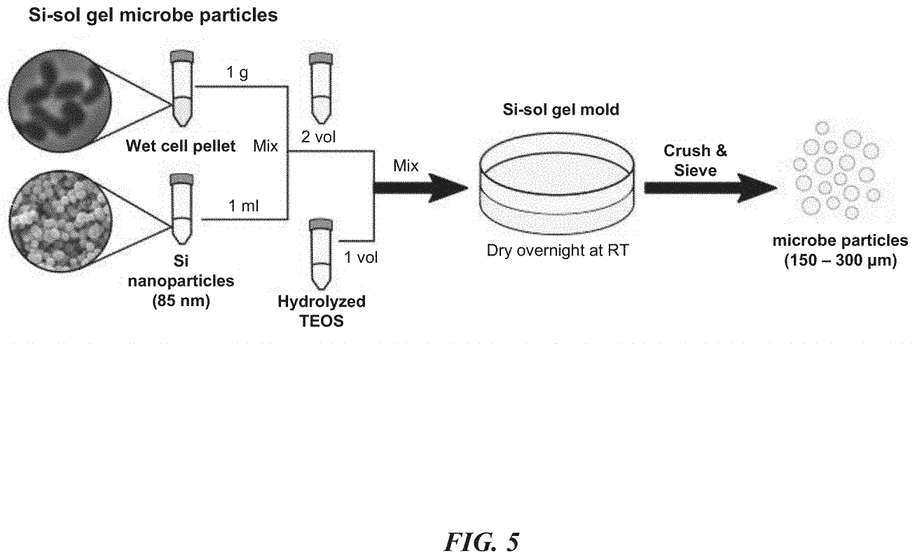

To apply A. nicotianae for efficient and scalable Sc and REE recovery, it is essential to immobilize the bacteria cells in a porous matrix with high chemical and mechanical stability. Porous silica was chosen for bio-adsorbent development given its high mechanical strength and resistance to acidic solutions (e.g., pH 3). Cells were encapsulated in a crosslinked silica nanoparticle (SNP) matrix in high density (0.5-2 wet g cells/ml) through a condensation reaction with hydrolyzed tetraethyl orthosilicate (TEOS). Since the high viscosity of the SNP:cell suspension precluded homogenous mixing with TEOS using a microfluidic approach, the SNP:cell:TEOS solution was mechanically mixed instead prior to gelling as a bulk solution (FIG. 5).

The microbe encapsulated SiO₂ gel (MESG) was overnight, crushed, and particles in the 150 to 300 μm size range were selected for downstream application in batch or a packed bed column format (FIG. 5). While the analysis was restricted to crushed particles, it is worth noting that the ability to fine tune the condensation reaction conditions via pH modulation enables the precursor solution to be cast into a mold with complex structures that provides the practicality and flexibility scale-up process scale-up and industry applications.

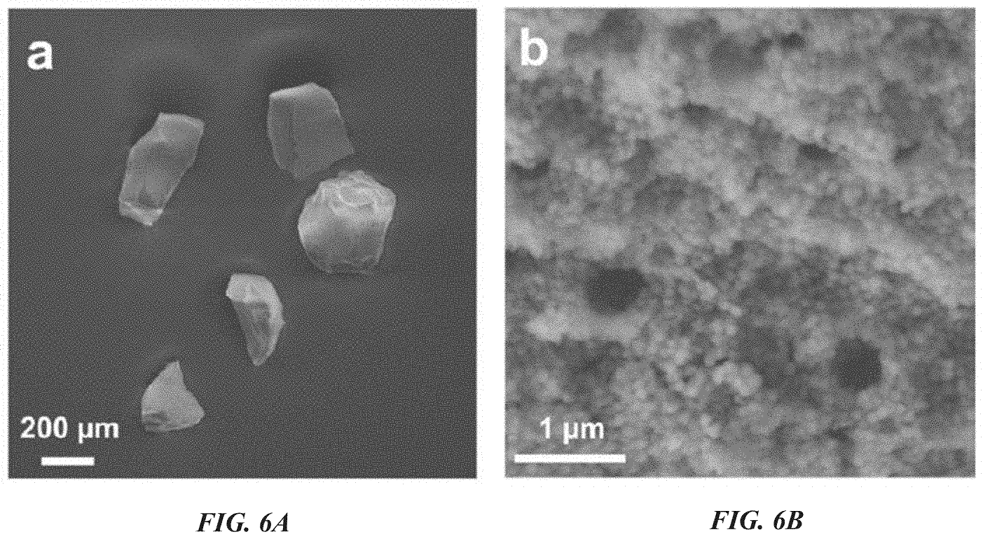

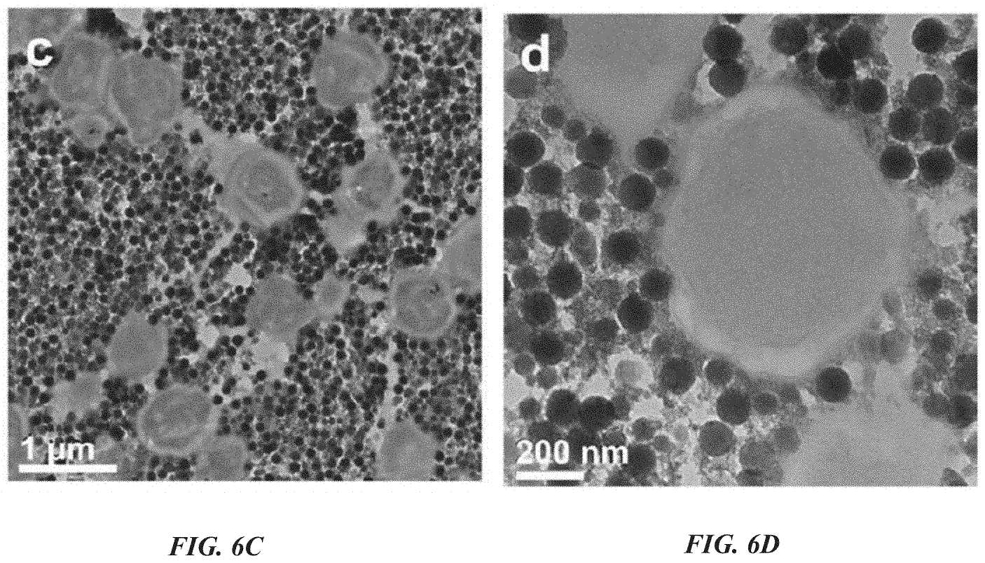

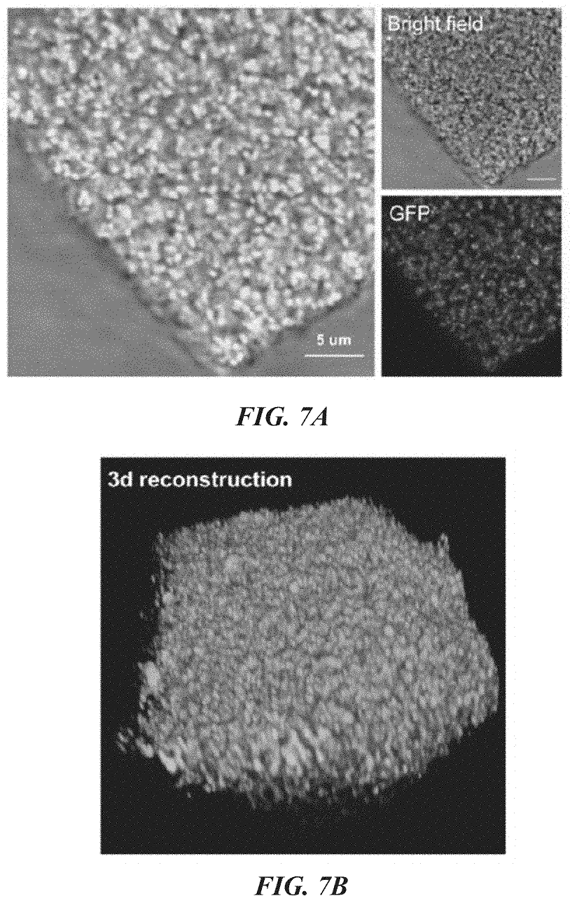

The MESG particle morphology, cell distribution, and porous structure were characterized using several complementary microscopy techniques. SEM imaging analysis revealed that the MESG particles are an irregular cuboid shape with lengths falling within the expected 150 to 300 μm size range (FIG. 6A). Higher magnification images of the particle surface showed evenly distributed holes on the surface of silica gel which are attributed to the loss of incompletely encapsulated cells (FIG. 6B). This data is consistent with a mechanism of physical entrapment in the silica gel matrix rather than chemical cross-linking like for SNPS. Interestingly, the individual SNPs are still visible in the SEM and thin section TEM images, suggesting that cross-linking with TEOS did not completely fill the gap between adjacent SNPs. This porous structure is sufficiently large to enable adsorbates to freely diffuse in and out the gels, given the small aqueous ionic radii for lanthanides of ˜0.25 nm [7] and a hydrodynamic radius of 0.37 nm for the eluent citrate, [8] but small enough to preclude the loss of 1 μm sized A. nicotianae cocci 191. Lastly, both confocal microscopy and TEM with thin-sectioned MESG particles indicated that the cells were densely and homogenously distributed within the microbe beads (FIG. 6C-6D: FIG. 7A-7B).

Collectively, these imaging results support the stable encapsulation of a dense population of A. nicotianae cells within the Si-sol gel matrix.

### Fabrication and Characterization of Microbe Encapsulated SiO₂ Gel

Batch adsorption experiments were conducted to evaluate the adsorption performance of the MESG particles and to determine the optimal cell density. A pH of 3 was chosen for assays given the limited Sc solubility above pH 4 and the stronger Sc complexes formed with hard ligands (i.e. carboxylic acids) compared to REEs on account of the smaller ionic radius and stronger Lewis acid character of Sc. It has been reported that lanthanide biosorption is significantly reduced when the solution pH is lower than 4 due to competition with protons for carboxylate functional groups.

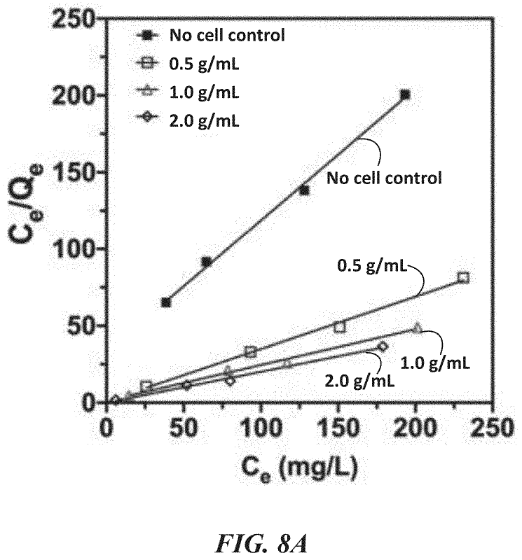

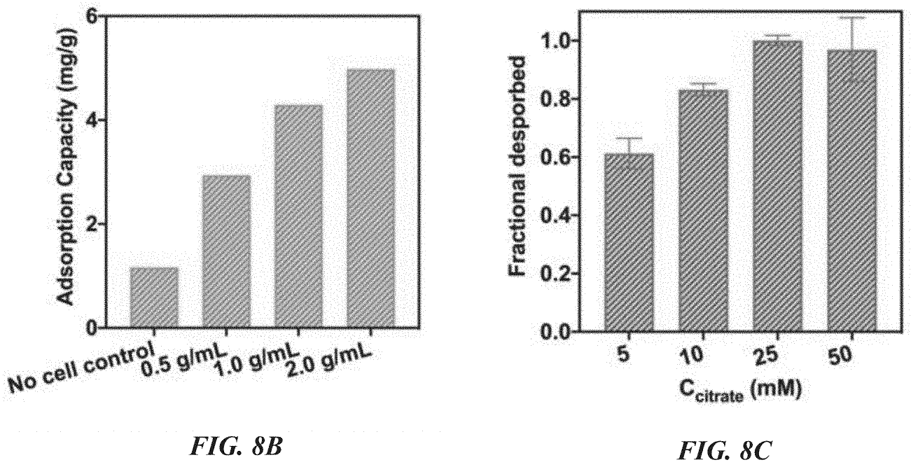

The batch adsorption data were well fit by the Langmuirisotherm model where monolayer adsorbates are assumed to be adsorbed onto a surface containing a finite number of adsorption sites (FIG. 8A). While higher cell loading increased the Sc adsorption capacity, the adsorption capacity was not proportional to the cell density above 1 g/ml (FIG. 8B). This suggests that a 1 g/ml density offers the optimal balance between cell loading and adsorption capacity. Next efficacy of sodium citrate (pH 6) was tested to desorb Sc ions and regenerate the MESG particles. At a volume ratio of 1:40 (gel:citrate), 25 mM citrate was required for complete Sc desorption (FIG. 8C). Lower citrate concentrations required larger volumes for regeneration (data not shown).

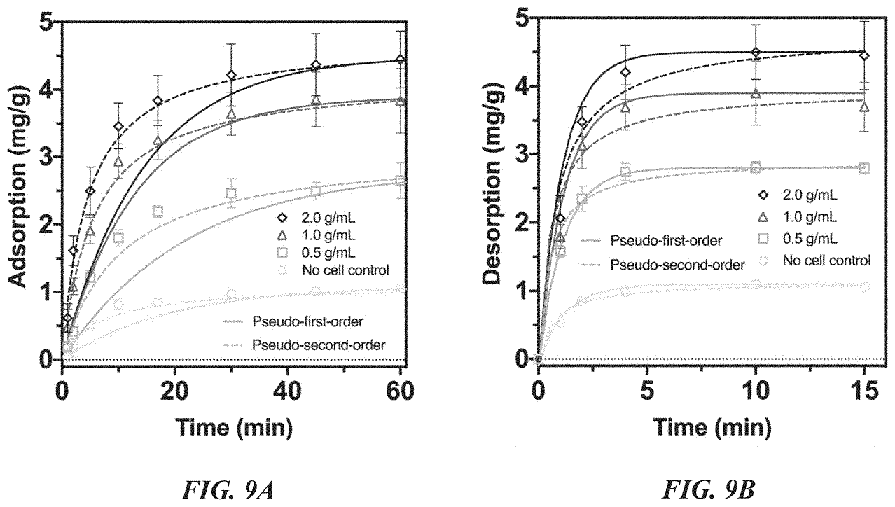

To further characterize the MESG particle function, Sc adsorption and desorption kinetics were assessed in batch reactions. The MESG particles were dispersed in 1 mM pH 3 Sc solution and the residual Sc concentration was measured as a function of time. Sc was rapidly adsorbed by MESG in the first 10 min incubation and then gradually reached equilibrium at about 30 min (FIG. 9A). Sc Desorption with 25 mM citrate (pH 6) occurred with even faster kinetics; equilibrium was reached with all cell loading densities within 10 min (FIG. 9B).

### Fabrication and Characterization of Microbe Encapsulated SiO₂ Gel

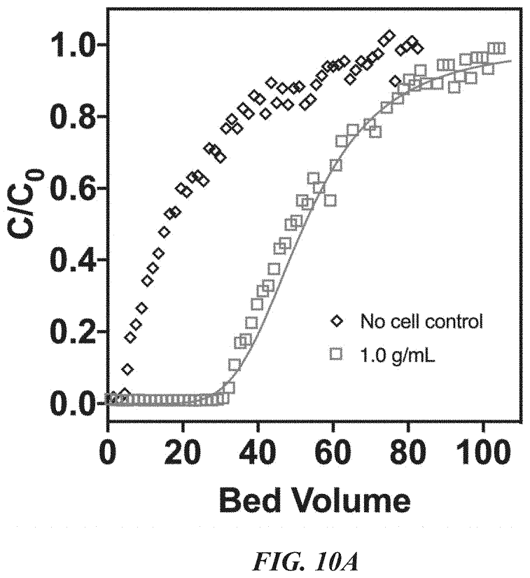

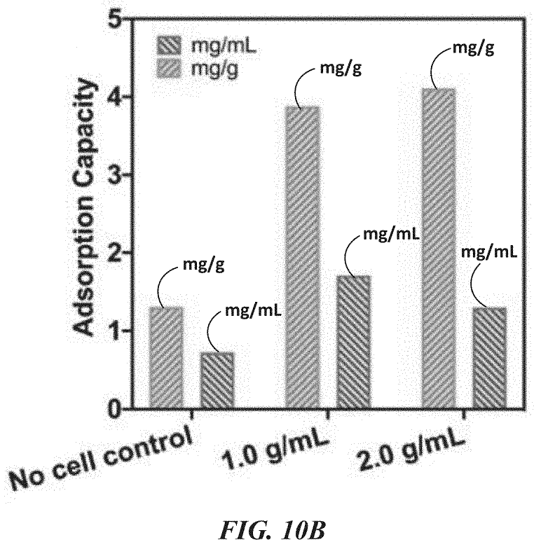

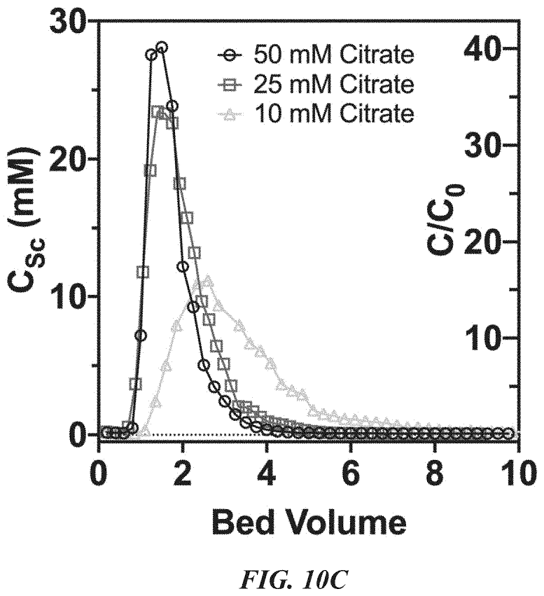

To test the efficacy of the microbe beads for Sc extraction under flow, fixed-bed columns were packed with the MESG particles and the influent breakthrough behavior was assessed with synthetic solutions containing 0.7 mM Sc at pH 3. Based on the results of the batch adsorption experiments, 1.0 g/mL and 2.0 g/mL gels were further selected as adsorbent candidates for fixed-bed column studies. The breakthrough points for 1.0 g/mL MESG particles occurred after 30.3 and 24 bed volumes, respectively, in contrast to cell-free MESG particles, where breakthrough occurred after only 3 bed volumes (FIG. 10A). Interestingly, for all three breakthrough curves, including the no cell control, the increase in Sc concentration in the effluent after initial breakthrough was slow compared to similar breakthrough experiments with REEs such as Nd. This phenomenon can be attributed to kinetically limited Sc adsorption onto the silica matrix. The major benefit of fixed-bed adsorption compared with batch adsorption is that the fluid exiting the column is free from desired adsorbate up to breakthrough point. Therefore, although pure silica (no cell control) gels exhibited a reasonable Sc adsorption capacity under batch conditions, the pure silica gels are not practical for a continuous column process at high flow rate.

In addition, the 1.0 g/mL gel packed column showed higher Sc adsorption performance than the 2.0 g/mL gel packed column, while greater capacity was observed by 2.0 g/mL gel in batch experiments (FIG. 10B). This inconsistency is mainly due to the difference of density of adsorbents. It is worth noting that the adsorption capacities of adsorbent are commonly reported in unit weight whereas adsorption capacity of a fixed-bed column is determined by the total volume of the packed adsorbent. It is not enough to predict column adsorption performance solely based on capacity per unit weight as the density of adsorbent varies depending on composition. As a result, the 1.0 g/mL gel was selected due to the highest column Sc adsorption capacity.

### Microbe Encapsulated SiO₂ Gel Column Reusability

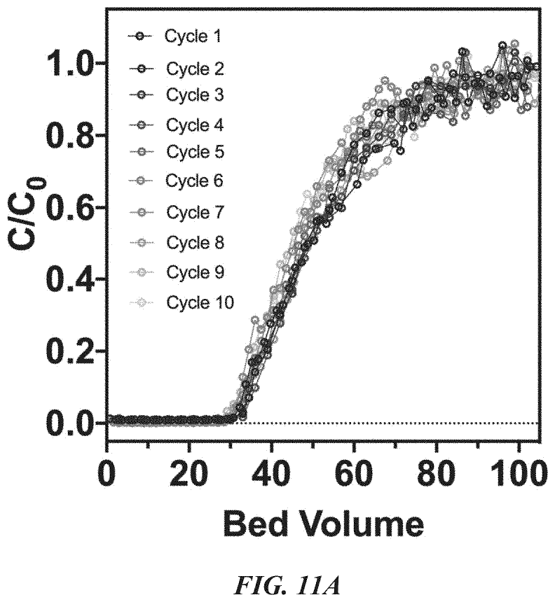

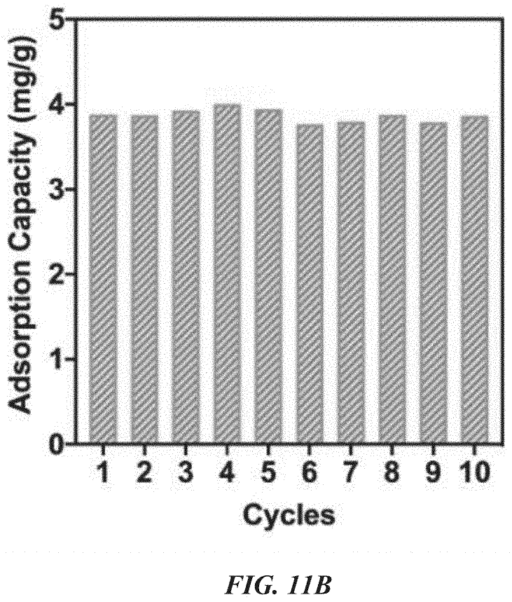

Adsorbent reusability is a key factor for economic feasibility of the adsorption process. In order to desorb Sc and enable MESG column reuse, the effect of citrate concentration on column desorption was investigated by washing Sc saturated columns with different citrate concentrations (10 mM, 25 mM, and 50 mM). A broad desorption peak was achieved using 10 mM citrate whereas sharp desorption peaks were with higher citrate concentrations with only a subtle difference observed between the 25 mM and 50 mM citrate desorption curves (FIG. 10C). As such, similar to the batch adsorption experiments, 25 mM citrate is sufficient to achieve a concentrated Sc solution. The ability of the MESG particle column to withstand multiple adsorption/desorption cycles was next tested using 0.7 mM Sc solution (pH 3, 10 mM glycine) as feed stock solution. After each adsorption process, the column was regenerated by 10 bed volumes of 50 mM citrate solution and reconditioned by 5 bed volume of pH 3 glycine solution. As shown in FIG. 6, at least 95% of the original adsorption capacity was maintained after 10 consecutive adsorption/desorption cycles (FIG. 11A-B). For each cycle, a feed of 0.7 mM Sc in 10 mM pH3 glycine was used for adsorption and 20 mL of 50 mM citrate was used for desorption. Confocal microscopy was used to examine the cell density of embedded cells after 10 adsorption/desorption cycles. The encapsulated cells remained homogenously distributed and appeared intact, based on SYTO 9 nucleic acid staining (Data not shown).

Collectively, these data support high Sc adsorption capacity and reusability for MESG particles with synthetic Sc only solutions.

Sc Extraction from a Synthetic Solution

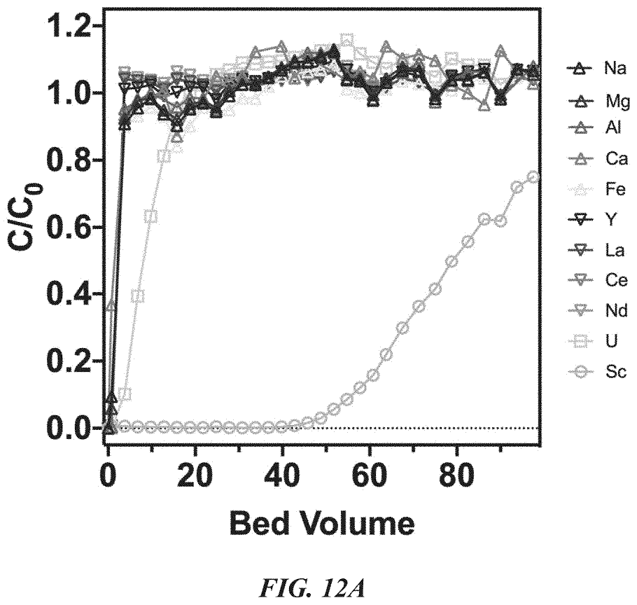

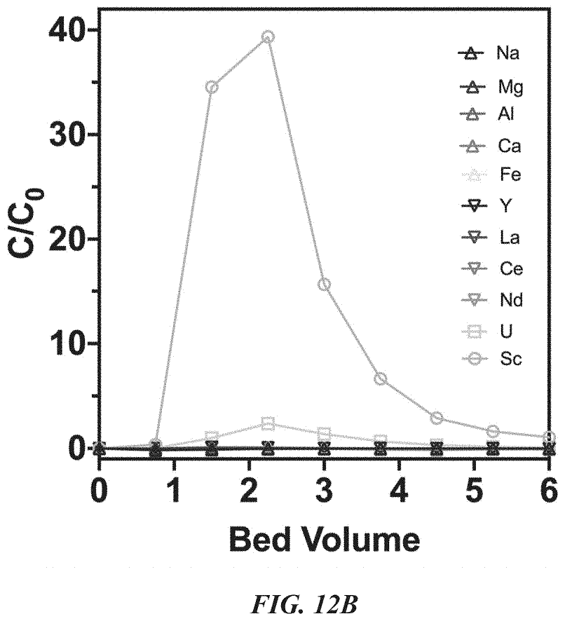

Industrial feedstock solutions, such as coal byproducts and bauxite residue, contain a high concentration of competing metal ions that could affect the Sc adsorption behavior. As a first approach to determine the effect of matrix elements on Sc adsorption efficacy, a breakthrough column experiment was performed using a synthetic solution containing 100 μM Sc, mM levels of major Non-REEs (Na, Mg, Al, Ca, and Fe) and total REEs (comprised of Y, La, Ce, and Nd), and a trace amount of the radionuclide uranyl (U)(10 μM). The ion composition of the synthetic solution is shown in Table 2. Scandium breakthrough occurred after 45 bed volumes while all other metal ions, with the exception of U, broke through within the first bed volume (FIG. 12A), indicating high selectivity for Sc against other ions. U exhibited a more gently sloped breakthrough curve relative to the other non-REE element in the synthetic solution, indicative of the higher binding affinity of U for cell surface sites compared to base metals and consistent with a prior report of U absorption by A. nicotianae at low pH (4).

Nevertheless, the 40+ difference in the breakthrough point for Sc compared to U and the observation that U concentrations exceeded the influent concentrations (C/C₀>1) following Sc breakthrough, which is a hallmark of competitive displacement (i.e., ion exchange), is suggestive of the higher cell surface affinity for Sc. To desorb the adsorbed Sc, the column was treated with 50 mM citrate (pH 6). At least 95% of the Sc content was recovered within 6 bed volumes and an enrichment factor of nearly 40 was observed for the most concentrated fractions (FIG. 12B). A slight concentration of U (˜2.5-fold) was observed as expected based on the breakthrough curve.

Overall, these results support the efficacy of MESG particles for the selective extraction of Sc from complex solutions.

TABLE 2
Ion Composition of the Synthetic Solution

| | Na | Mg | Al | Ca | Sc | Fe | Y | La | Ce | Nd | U |
|---|---|---|---|---|---|---|---|---|---|---|---|
| Concentration | 700 | 15 | 12 | 22 | 0.11 | 7.2 | 0.26 | 0.22 | 0.45 | 0.22 | 0.01 |
| (mM) | | | | | | | | | | | |

Sc Extraction from a Leachate

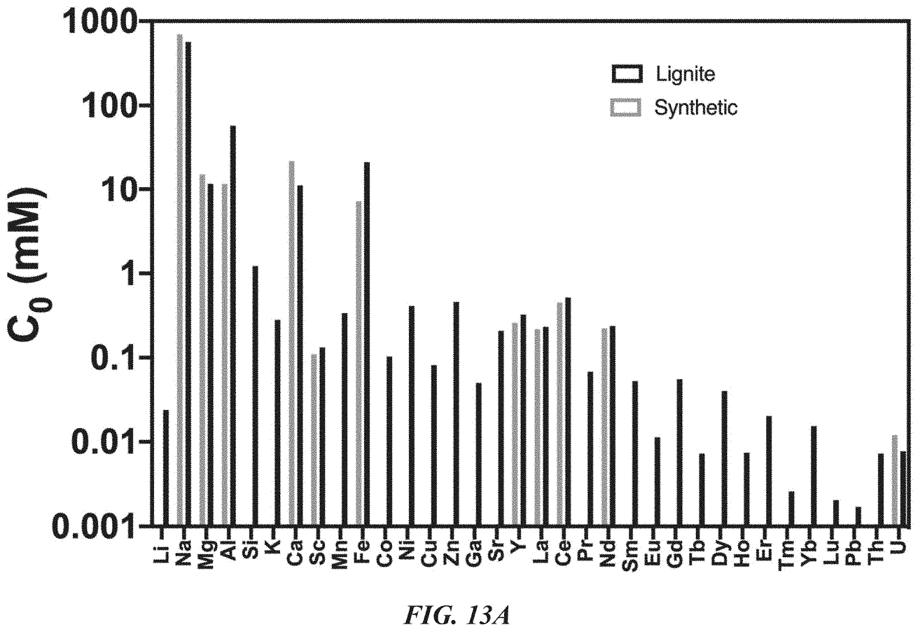

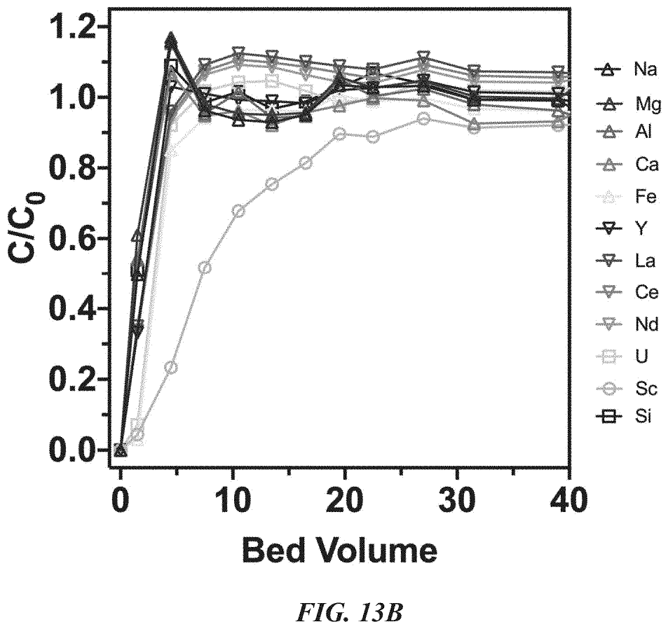

To test the performance of the MESG particles for Sc extraction with a real feedstock, a breakthrough column experiment was performed with full-strength leachate (pH 3) prepared from lignite coal at a pilot plant operated by the University of North Dakota. The leachate contained 134 μM Sc, 1.5 mM total REEs, and Na, Mg, Al, Ca, and Fe at concentrations greater than 10 mM (FIG. 13A). In addition to the >3-fold higher concentrations of Al/Fe compared to the synthetic leachate, the lignite leachate contained significant levels of transition metals (e.g., Zn, Ni, Mn), the entire lanthanide series (except Pm), and a trace level of the radionuclide Thorium (h)(FIG. 13A). With the unmodified lignite leachate. Sc breakthrough was observed after only a few column volumes (FIG. 13B). The earlier breakthrough compared to the synthetic solution is likely attributed in part to the higher concentration of the hard cation Fe³⁺ in the lignite solution, which is expected to be a strong competitor for hard ligands, such a cell surface carboxylate functional groups.

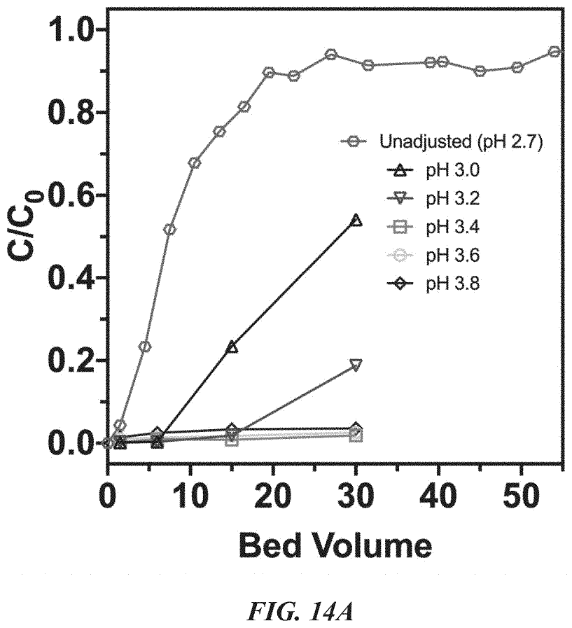

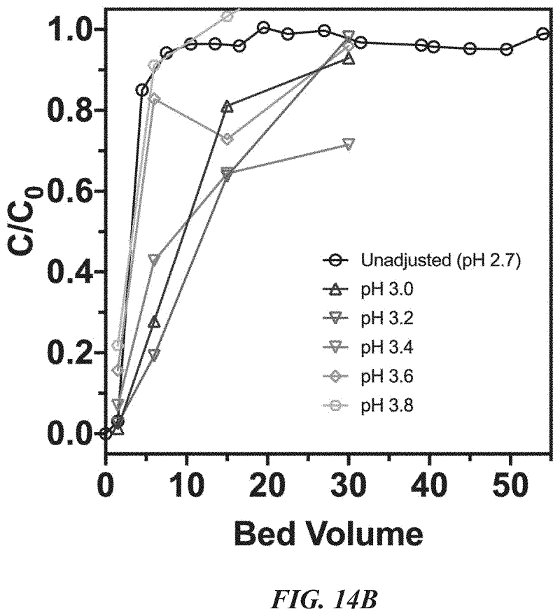

To improve the Sc extraction performance, lower expected solubility of Fe hydroxides compared to Sc/REE hydroxides in the pH 3-4 range was leveraged. The pH of the lignite feedstock was incrementally increased from pH 3 to 3.8. Quantification of the metal ion concentration before and after pH adjustment revealed a significant reduction in the Fe concentration and minimal if any reduction in the concentration of Sc, REEs, or other major elements. To test the effect of reduced Fe concentration on Sc recovery, each pH adjusted solution was adjusted back to pH 3, to facilitate direct comparison (i.e., eliminate pH as a variable), and Sc and Fe breakthrough was assessed over 30 bed volumes (FIG. 14A-14B).

Importantly, REE breakthrough was proportional to the pH adjustment step, with Sc failing to breakthrough after 30 bed volumes with lignite solutions that had been previously adjusted to 3.4, 3.6, and 3.8 (FIG. 14A). Conversely, the Fe breakthrough activity was inversely correlated with the pH of the adjustment step (FIG. 14B). Collectively, these data suggest that the Fe³⁺ concentration is the major driver of Sc extraction efficacy.

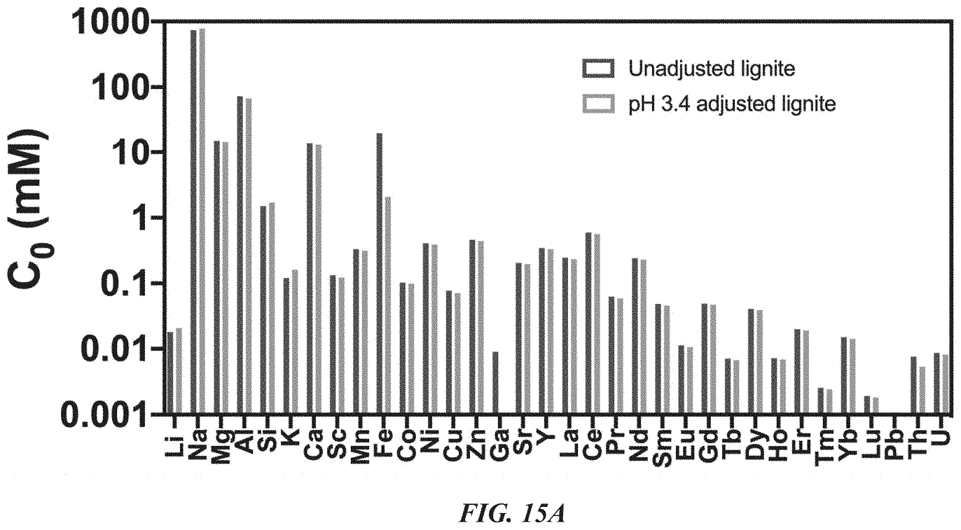

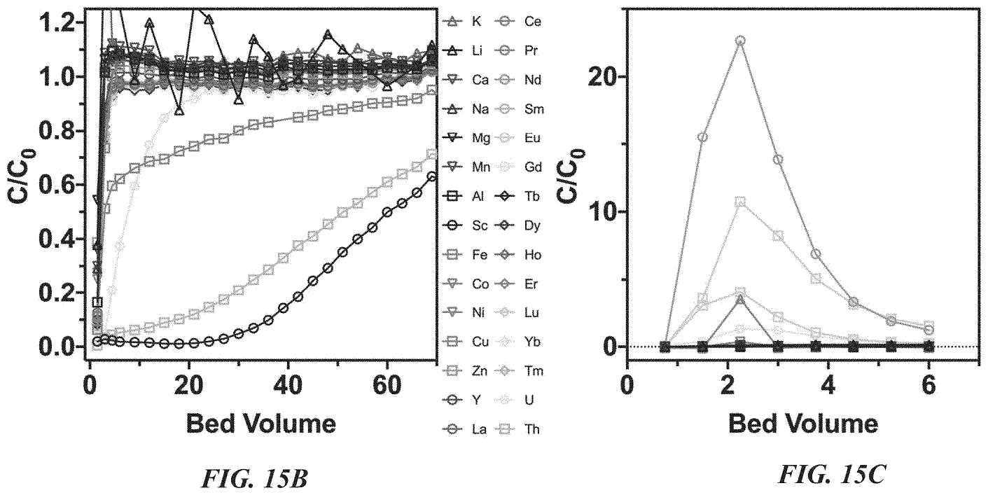

Lastly, the effluent concentrations for each metal ion in a pH 3.4-adjusted lignite leachate (FIG. 15A) were quantified over 70 bed volumes. Sc breakthrough occurred after 30 bed volumes, whereas the majority of other elements broke through within the first bed volume as part of the void volume (FIG. 15B). The adsorption behavior for U mirrored that of the synthetic leachate. By using full strength lignite leachate, the breakthrough behavior of Th was quantified. Th breakthrough occurred almost immediately but exhibited a gentle-sloped breakthrough curve that resembled the shape of Sc breakthrough curve as a C/C₀ of 1 was approached. It is contemplated that this breakthrough profile is a result of Th sorbing to the Si-sol gel matrix rather than A. nicotianae cells. As such, the adsorbed Th can likely be separated from Sc using a gentle acid wash step prior to Sc desorption.

Using 50 mM citrate solution, over 95% of the adsorbed Sc was desorbed within bed volumes, with an average enrichment factor of 10.9 and an enrichment factor of 23 for the most concentrated fractions (FIG. 15C-15D). While a 10-fold concentration of Th was observed, Th only minimally impacted the purity of the eluted Sc solution given its low starting leachate concentration (>10-fold lower concentration compared to Sc). Importantly, a separation factor of 355 was observed for Sc over total REEs. We found <0.6% of the lanthanides were coextracted; Sc constitutes 7.1% of the total REEs in the leachate and this number increased to 96.4% in the desorbed citrate solution after a single adsorption-desorption cycle. These results highlight the ability of the MESG biosorbent to selectively concentrate Sc from lignite leachate.

### Two-Stage Sc, REE+Y Extraction Process

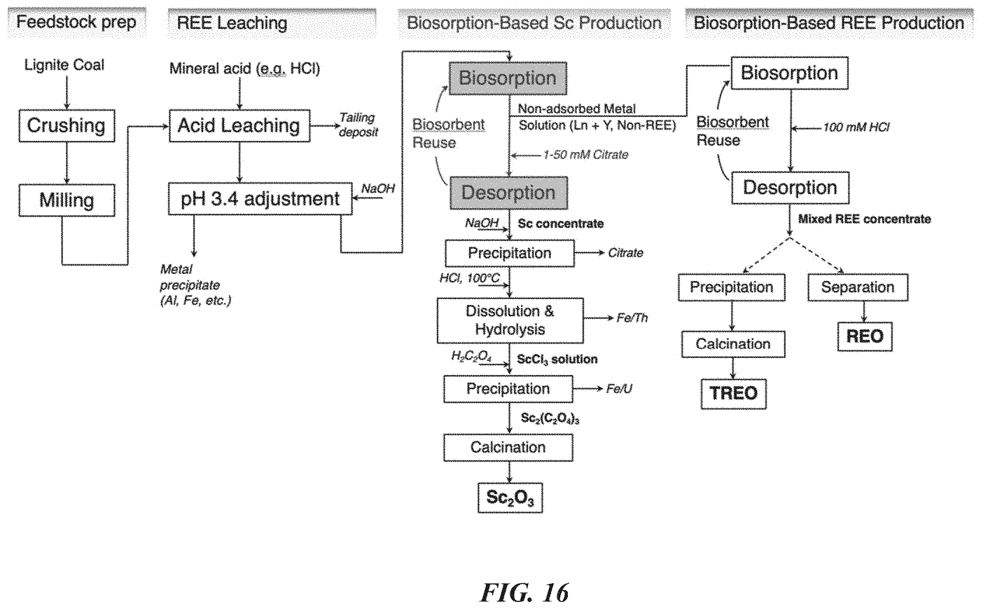

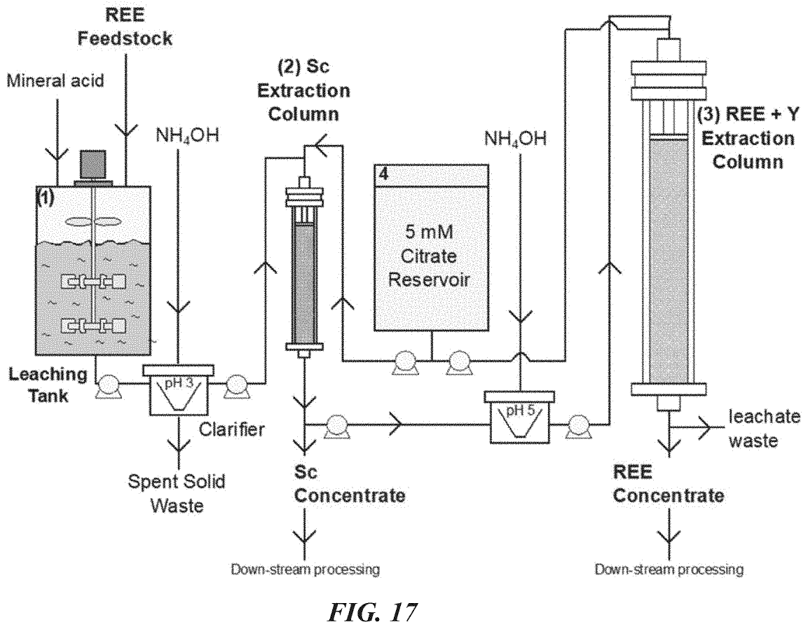

The high Sc extraction efficiency and low REE recovery at low pH (3) support the potential of a two-step biosorption procedure to achieve Sc separation and total REE recovery; an initial biosorption step at pH 4 to separate Sc from REEs followed by pH adjustment to 5 and subsequent separation of total REEs from non-REEs. The two-stage process is outlined in FIGS. 16 and 17 and described below in detail.

Following an acid leaching step to produce a pregnant metal solution from the lignite coal, the pH is adjusted to 3.4 and the leachate is passaged over a microbe particle column where Sc is selectively adsorbed onto the bacterial surfaces. Weakly adsorbing LN+Y and base metals, which are present in significantly higher concentration relative to the adsorptive surface sites in the microbe bead resin, are collected in the flow through. Immediately prior to Sc breakthrough, the inlet feedstock flow is shut down and Sc is desorbed by circulating a small volume of citrate solution (5 mM, pH 6). The Sc-depleted flow through solution is adjusted to pH 5 to precipitate Al and Fe impurities and passaged over a second microbe resin column for selective LN+Y adsorption, while weakly adsorbing alkaline earth and d block metals are discarded in the flow-through. Immediately prior to REE breakthrough, the microbe resin subjected to a citrate circulation step (5 mM, pH 6) to desorb and concentrate LN+Y. Both extraction columns can be reused multiple times with only minimal loss in Sc/REE adsorption capability. It is anticipated that an analogous two-step biosorption procedure will apply to other Sc/REE+Y-containing feedstocks such as bauxite residues (i.e., red mud) produced form Al extraction operations and coal fly ash.

Process flow diagram of FIG. 16 shows a biosorption-based Sc-extraction process from lignite coal. Similar to other hydrometallurgical processes, such as solvent extraction and ion exchange, the application of biosorption for Sc recovery from coal feedstocks requires pre-processing (e.g., mining, crushing and milling), solubilization of solid feedstocks through leaching, and pH-adjustment to facilitate biosorption. In such scheme, the goal of the biosorption step would be to selectively recover Sc in an initial extraction step while allowing for REE separation from non-REEs in a downstream extraction step. Additional downstream procedures such as precipitation and filtration may be required to further remove the impurities (e.g., Th and U) and produce a Sc2O3 product. [22] In a traditional downstream purification process, for example, the Sc concentrate along with impurities (Th, U, Fe and Si) could be precipitated by NaOH and filtered to recycle the citrate. Then, the filtration cake could be digested at pH 4 (HCl) and 100° C. to selectively redissolve Sc. Subsequently, the redissolved Sc will be precipitated with oxalic acid to isolate it from co-dissolved U. [22] As such, MESG particles provide an effective means to separate and concentrate Sc from lanthanides and non-REEs starting from unconventional low-grade feedstocks, transforming a highly complex mixture solution into high-purity Sc concentrates that can be fed to various traditional hydrometallurgical processes.

### Selective Sc Adsorption in Batch Experiments

To test for selective Sc extraction, batch adsorption experiments were performed with the MESG biosorbent in synthetic solutions containing equimolar concentrations of Sc, Al, Fe(III), Y, and Nd. Aluminum and Fe are abundant in Sc-bearing feedstocks [11] and known to complex strongly with cell surface functional groups. [12, 13] Neodymium and Yttrium are abundant and green energy critical REEs [14] that are representative of early and later lanthanides, respectively, in terms of their atomic radii. [15]

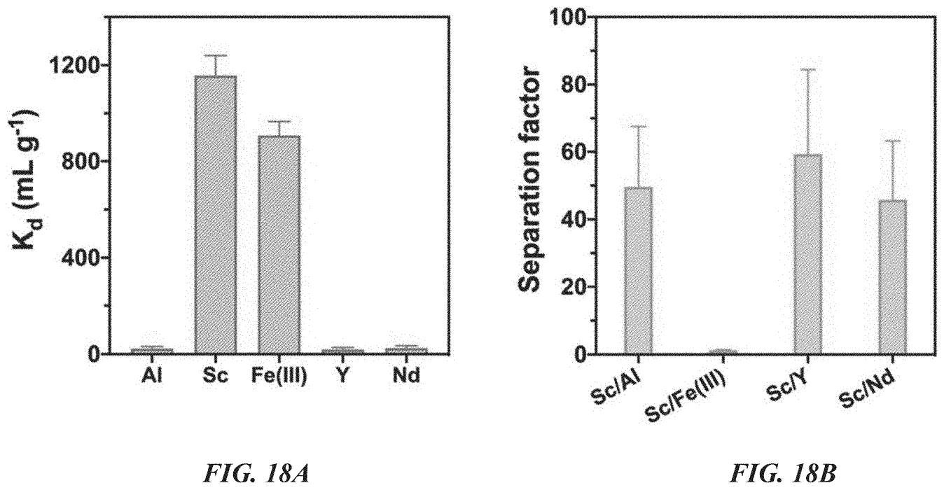

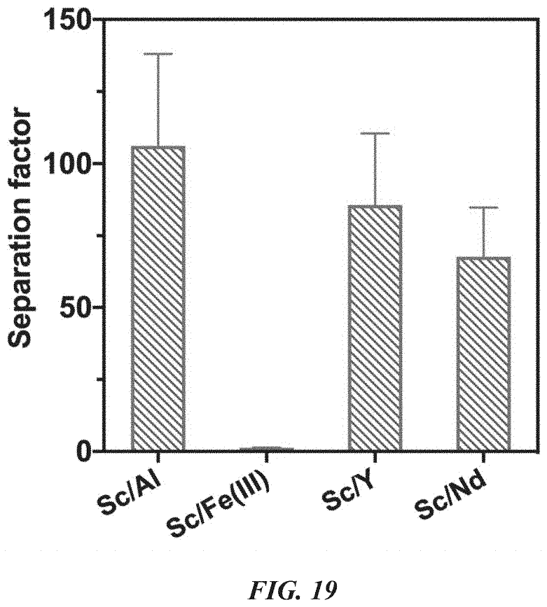

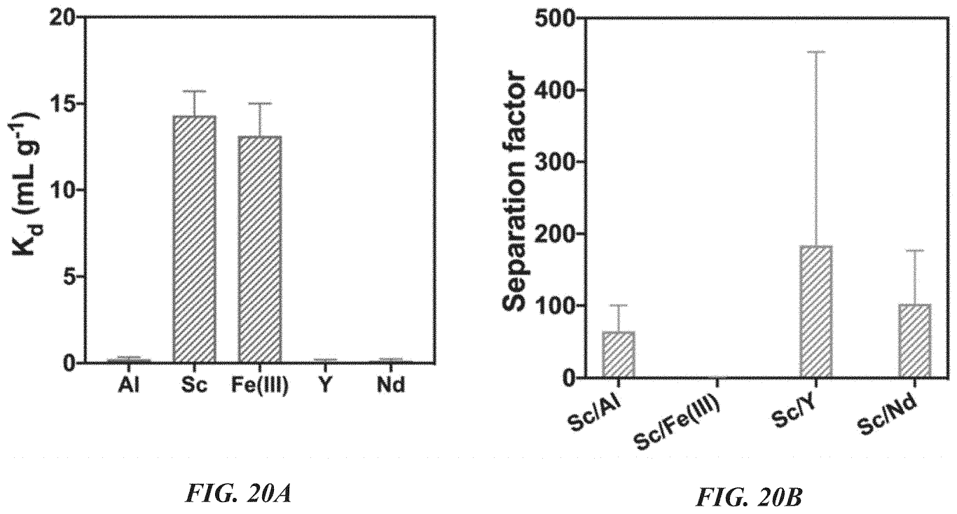

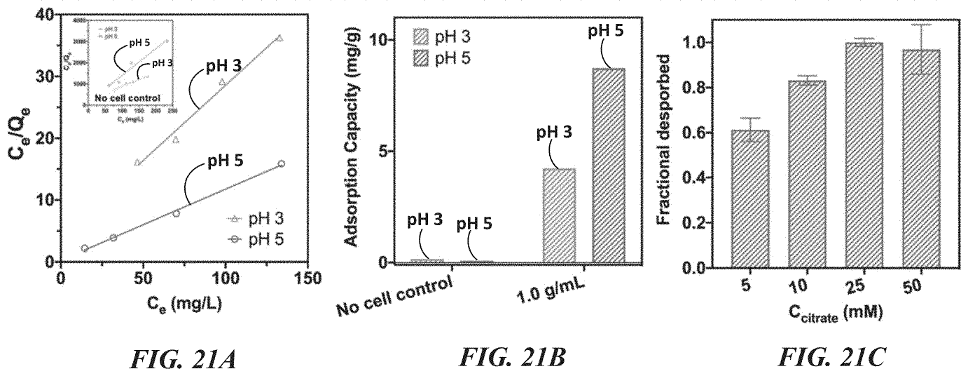

The MESG biosorbent displayed high Sc selectivity, consistent with the behavior of the unencapsulated cells (FIGS. 18A-18B). Effective separation of Sc from Y and Nd was achieved in a single adsorption step with separation factors of 86 and 68 achieved for Sc/Y and Sc/Nd, respectively (FIG. 19). It is worth noting that selectivity for Sc over Y and Nd was also observed for cell-free silica (FIGS. 20A-20B), albeit with much lower total adsorption compared to MESG (FIGS. 21A-21C). The selectivity of the MESG biosorbent for Sc is likely attributed to the ability of cell surface hard ligands (i.e., phosphate groups) and silanol groups on silica to form strong complexes with Sc(III) ions, which has a smaller ionic radius and stronger Lewis acid characteristics compared to the lanthanides. [16, 17].

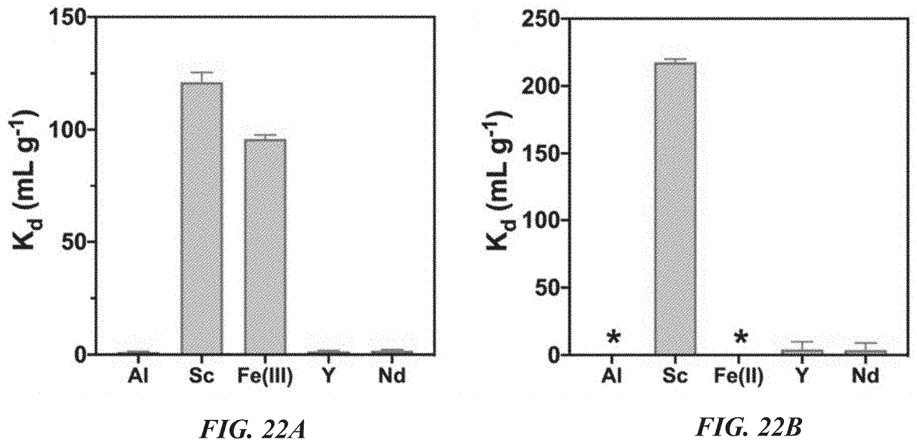

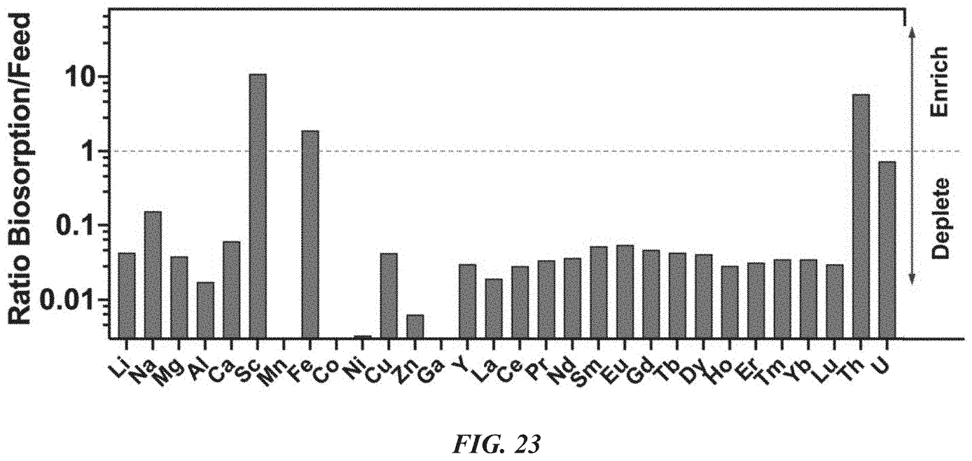

For non-REEs, the MESG biosorbent showed high Sc selectivity over Al but low selectivity against Fe(III), a behavior that was also observed for unencapsulated cells (FIGS. 18A-18B). Competitive adsorption between Sc and Fe(III) has been widely reported as these trivalent cations possess similar Lewis acidity and ionic radius (0.745 Å for Sc(III) vs 0.645 Å for Fe(III)). [11, 12, 18-21]. Similarly, Fe co-extraction was reported by bisphosphonate grafted porous silicon and supported ionic liquid adsorbent. [19-21] Fe(III) was replaced with Fe(II) in the multi-element batch adsorption, and negligible Fe adsorption was observed (FIGS. 22A-22B), confirming that the Fe(III) ion is the major competitor for Sc adsorption and should be removed or reduced to Fe(II) before Sc extraction. Together, these data indicate that silica sol gel encapsulated A. nicotianae retains the Sc selectivity of unencapsulated cells.

### Example 2: Scalable Microbe Encapsulated PEG Gel Sc Selective Exaction from Coal by-Product

The following example describes an efficient and scalable Sc and REE recovery process compatible with industrially relevant flow rates. While this example specifically describes Sc separation, it is contemplated that the methods described herein can be similarly applicable to any REE, including La, Ce, Pr, Nd, Pm, Sm, Eu, Gd, Tb, Dy, Ho, Er, Tm, Yb, Lu, and/or Y.

### Materials and Methods

Chemicals and strains. The purity of metal salts was as follows: scandium(III) chloride hexahydrate (99.999%), aluminum chloride (99.99%), ferrous chloride tetrahydrate (99.99%), Neodymium(III) chloride (99.99%), Magnesium chloride hexahydrate (99%), and Yttrium(III) chloride hexahydrate (99.9%).

Growth of REE-absorbing bacteria. Arthrobacter nicotianae (ATCC 15236) was grown overnight, subcultured using a 1:50 dilution, and then grown in LB media for 24 hours at 30° C. Arthrobacter nicotianae cells were harvested by centrifugation at 7,000 g for 7 min, washed once with 0.9% (w/v) NaCl saline solution, decanted, and stored at 4° C. until use.

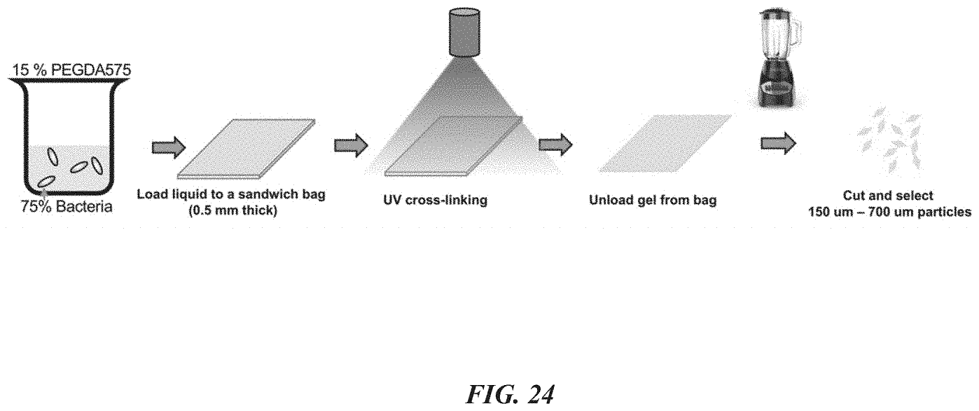

Microbe Encapsulated PEG Gel (MEPG) Particle Fabrication. The MEPG particle fabrication method is summarized in FIG. 24. Free radical polymerization was carried out to encapsulate cells within polyethylene glycol diacrylate (PEGDA) hydrogel. Specifically, a polymer precursor solution was prepared using 99% w/w PEGDA monomer (Mn 575; Sigma Aldrich) with 1% w/w TPO-L photoinitiator (2,4,6-Trimethylbenzoylphenyl phosphinic acid ethyl ester; Rahn AG). The polymer precursor was then mixed at 15% w/w with a concentrated cell pellet (85% w/w) containing ˜1×10¹¹ cells/mL. The resulting cell/polymer precursor solution was then purged with N₂ for 10 min and transferred into transparent sandwich bags (11 cm×10 cm, 5 mL), which were immediately exposed to UV (10 mW/cm² at 365 nm) for 300 s to polymerize the hydrogel sheet comprised of PEGDA and cells. The polymerized sheet was chopped using a wireless electric small food processor & food chopper (10 Oz, 150 Watts, Kocbelle). The resulting microbe encapsulated PEG gel (MEPG) particles with desired sizes were selected by sieving and stored in DI-water at 4° C. until further use.

Microscopic Characterizations. For SEM analysis, MEPG particles were characterized by scanning electron microscopy (Thermo Scientific Apreo 2 SEM, USA) at 5 kV. Particles were washed with DI water for 3 times and dried at room temperature for 48 h. The dried samples were scanned under SEM at 300, 1000, 2500, and 10000 magnifications.

Breakthrough Column Experiments by Synthetic Solution. Econo-Column glass chromatography columns (Bio-Rad; 50 cm×0.7 cm, 20 mL) were used for continuous flow REE recovery experiments. Each column was filled with DI-water before adding MEPG particles gravimetrically. Approximately 100 mL DI-water was pumped through the column at 2.5 mL/min to compress the particles and more MEPG particles were added. The process was repeated until the entire column was packed. Single metal element synthetic solutions were prepared to evaluate the metal ion adsorption behavior of the column at different operation conditions. Scandium (50 mM, Sc) stock solution was prepared by dissolving scandium (III) chloride hexahydrate in 1 mM HCl. The stock solution was diluted in glycine buffer (pH 3.0, 10 mM). Prior to adsorption, the columns were conditioned with at least 5 bed volumes of DI-water. Subsequently, the feedstock solution was pumped through the column at 2.5 mL/min unless otherwise specified. The influent Sc concentrations were prepared in glycine buffer and ranged from 0.24 to 2.2 mM Sc. The column effluent was collected in 9.5 mL aliquots and analyzed by using Arsenazo III assays and/or ICP-MS. To desorb REE and enable column reuse, at least 5 bed volumes of sodium citrate (pH 6.50 mM) were passed over the column before reconditioning with 5 bed volumes of DI-water. In between experiments, columns were stored in DI-water at room temperature. Dry gel weights and bed void fractions were measured by removing the MEPG particles from the columns and drying at 65° C. for 7 days. The REE adsorption capacities of the fix-bed columns were calculated via mass balance, as follows:

$$q = \frac{Q {C}_{0} {\int }_{t = 0}^{t = ∞} 1 - \frac{C}{{C}_{0}} d t - ɛ \frac{π {D}^{2} L}{4} {C}_{0}}{V} \qquad ( 8 )$$

where q is the adsorption capacity (mg/L), Q is the feed flow rate (mL/min), C₀ is the feed stock REE concentration (mg/mL), C is the effluent REE concentration (mg/mL), D is the column diameter (cm), L is the bed height (cm), ε is the bed void fraction (cm³ void/cm³ bed), V is the volume of adsorbent (L), and t is time (min). The integral portion of the equation was numerically calculated using Excel. The void fraction (e) of the fully packed bed (100±5% of the total volume) was determined by analyzing the total column weight (wet) and dried column weight. Breakthrough column modeling is described in the supporting information.

Leaching of lignite coal. North Dakota lignite coal was sourced from an outcrop of the H-Bed seam in the Harmon-Hanson coal zone in Slope County, N. Dak. (Sample 6A-2), with a particle size distribution of 20-100 US mesh, a total REE content of 634 ppm (dry whole coal basis), and a Sc content of 27 ppm. Leaching of the dried pre-combustion lignite was conducted as previous described and the post-leaching pH was adjusted to pH 2.7-3.0 by adding 1 M NaOH for storage. To remove excess Fe, the leachate was adjusted to pH 3.4 by adding 1 M NaOH solution. Approximately 7 mL of 1 M NaOH was used per 100 mL leachate for the entire pH adjustment process. Precipitates were removed by vacuum filtration (0.22 μm) and the pH of lignite leachate was adjusted to pH 3.0 by adding 1 M HCl (0.4-1 mL HCl per 100 mL leachate) for column adsorption study and long-term storage.

Column breakthrough experiments by lignite. A MEPG (Arthrobacter nicotianae) particle-filled column was used for pH 3.0 lignite breakthrough experiments for Sc selective extraction. The column effluent was collected in 9.5 mL aliquots and analyzed by using ICP-MS. Columns were pre-conditioned with DI-water for at least 5 bed volumes prior to passing the lignite solution through the column at a rate of 2.5 mL/min. Adsorbed metals were desorbed by pumping citrate (50 mM, pH 6) through the columns. Metal concentrations were quantified using ICP-MS.

Pure water flux. Pure water fluxes were measured by using columns packed with different particle sizes to a height of 46 cm. DI-water was constantly pumped onto the top of column to maintain a liquid level of 55 cm and the volume of DI-water that flowed through the column in 1 min was recorded.

Pressure drop modeling. Ergun equation:

$$\frac{Δ P}{L} = \frac{150 u {μ ( 1 - ɛ )}^{2}}{{D}_{P}^{2} {ɛ}^{3}} - \frac{1.75 ρ {u}^{2} ( 1 - ɛ )}{{D}_{P} {ɛ}^{3}} \\ {f}_{p} = \frac{150}{G {r}_{p}} + 1.75$$

$${f}_{p} = \frac{Δ p}{L} \frac{{D}_{p}}{ρ {v}_{s}^{2}} ( \frac{{ɛ}^{3}}{1 - ɛ} )$$

where ΔP is the pressure across the bed (Pa), L is the height of the bed (m), u is the superficial velocity (m/s), μ is the viscosity of fluid (Pa S), ε is the void fraction of the bed, ρ is the density of fluid (kg/m³), D_{P} is the equivalent spherical diameter of the particles (m). The void fraction was determined by analyzing the total column weight of a DI-water fill column and a wet gel filled column.

### Results

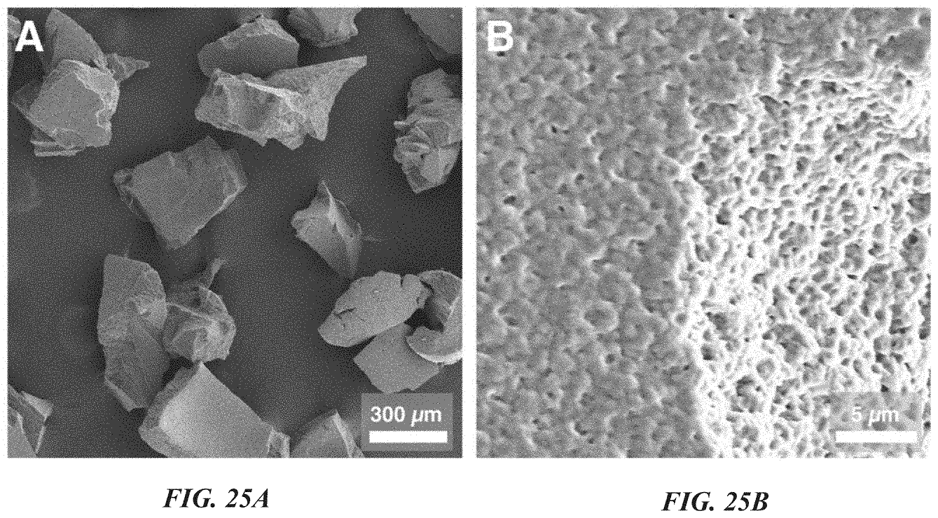

Fabrication and characterization of microbe encapsulated PEG gel (MEPG). Arthrobacter nicotianae cells were directly encapsulated in PEG gel through a scalable encapsulation method (FIG. 24). SEM imaging analysis revealed that the particles are an irregular cuboid shape with lengths falling within the expected 150 to 300 μm size range (FIG. 25A). Higher magnification images of the particle surface showed evenly distributed holes on the surface of silica gel that are likely attributed to the loss of partially encapsulated cells during sample preparation (FIG. 25B).

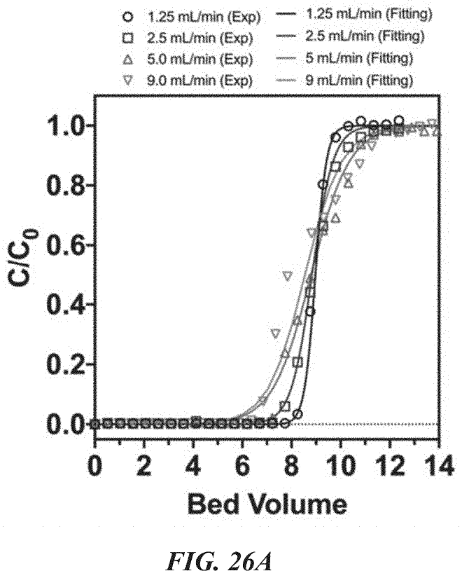

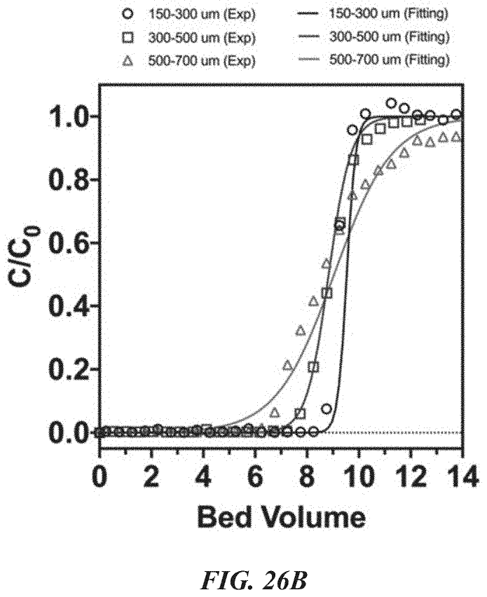

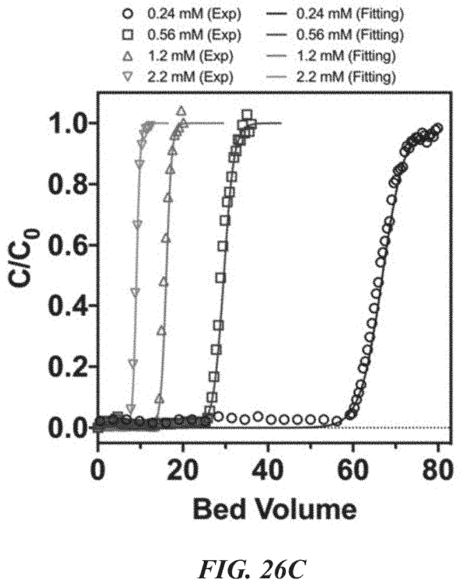

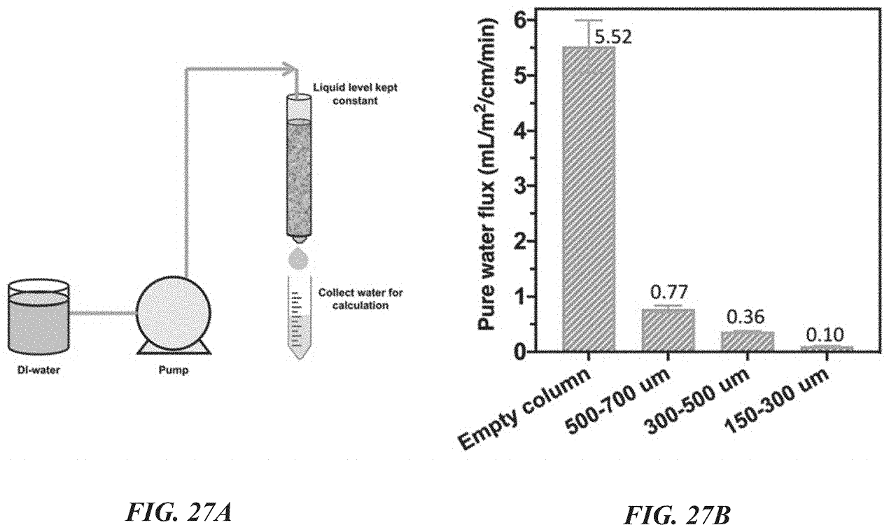

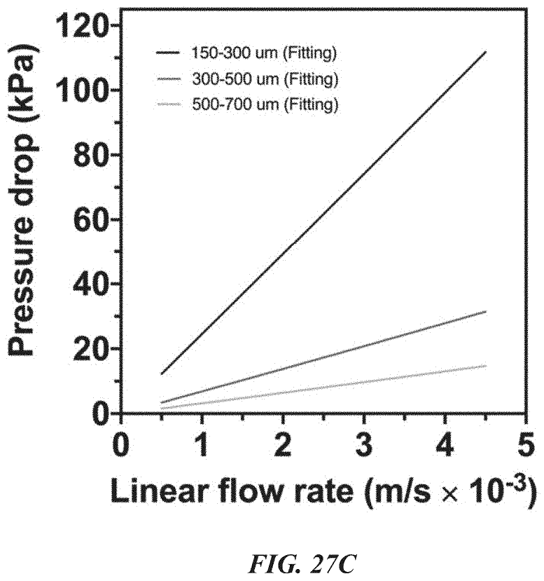

Effect of particle size. The particle size of the MEPG is a critical parameter in column operation. Although smaller particle sizes enable a higher mass-transfer rate, larger particles cause less pressure drop and thus higher throughput. The effect of particle size was studied by loading MEPG with different particle sizes in 20 mL columns (18 mL of adsorbents) which were tested at a constant flow rate of 2.5 mL/min with Sc concentration of 2.2 mM. As shown in FIG. 26A, breakthrough curves for all three different particle sizes show the typical sigmoidal shape, with smaller sizes exhibiting steeper slopes after breakthrough. When the particle size was increased from 150-300 μm to 300-500 μm, the Sc breakthrough point only decreased from 8.9 bed volumes to 7.7 bed volumes. When the particle size was further increased to 500-700 μm, the Sc breakthrough point was reduced considerably to 5.7 bed volumes. Smaller adsorbents likely enhanced adsorbate diffusion due to the shorter intra-particle diffusion depths. In contrast, the larger particles allow higher flow throughput compared with smaller particles. In a pure water flux tests, the 150-300 μm column only achieved a flux of 0.1 mL/m²/cm/min (FIG. 27A-27C), which is 3.6 times lower than the flux obtained by the 300-500 μm particles. However, only a 2-fold higher water flux was achieved when the particle size was further increased (500-700 μm). Given the trade-off relationship between mass-transfer-rate and flux, the 300-500 μm was selected for further investigation.

Effect of flow rate. Although a higher flow rate is usually preferred for higher throughputs, higher flow rates also cause higher head loss, resulting in a higher energy cost for column operation. In addition, it is desirable to allow absorption columns to be operated at a flexible flow rate range to accommodate the fluctuation of other parameters such as temperature, pressure, feed concentration, pH and viscosity. Therefore, fixed-bed columns are commonly operated at linear flow rate of 0.001-0.004 m/s. Hence, the effect of flow rate on column performance was investigated by using an 18 mL 300-500 μm MEPG packed column and passing pH 3.0 solution containing 2.2 mM Sc at different linear flow rates. As shown in FIG. 26B, higher flow rates resulted in earlier breakthrough due to decreased contact time. Such insufficient contact time also causes decreased adsorption capacity at higher flow rates. Within the low flow rate range of 0.54×10⁻³ m/s to 1.07×10⁻³ m/s, adsorption capacity remains unaffected. However, a 5-10% adsorption capacity lost occurred when the flow rate was further increased to 2.14×10⁻³ m/s to 3.9×10⁻³ m/s. Nevertheless, the 46 cm height column tested is still significantly shorter than an industrial scale column, where bed utilization efficiency will be improved as the unused bed portion will become a much smaller fraction of the overall bed length. Therefore, our results suggest that 300-500 μm MEPG adsorbents are readily compatible with industrially relevant flow rates.

Effect of concentration. The effect of the Sc concentration in the feed solution was investigated using synthetic solutions containing 0.24, 0.56, 1.2, and 2.2 mM of Sc, respectively (FIG. 26C). The variation in the initial Sc ion concentration extensively affected the breakthrough curve under the conditions of column bed depth of 46 cm and a flow rate of 2.5 mL/min. The breakthrough points were extended from 7.7 bed volumes to 58.8 bed volumes when the Sc concentration decreased from 2.2 mM to 0.24 mM, suggesting that a higher Sc concentration saturated the MEPG bed quicker than a lower concentration at the same flow rate. In addition, it was found that the column adsorption capacity declined from 808 to 706 mg/L with a decrease in Sc concentration in the range of 0.24 to 1.2 mM, which may be explained by the smaller driving force (concentration difference) for mass transfer. A larger Sc concentration difference between the absorbent and the solution offers a higher driving force for the biosorption process. A higher Sc concentration also generated sharper breakthrough curves for the same reason. However, column capacity did not further increase when the Sc concentration increased from 1.2 mM to 2.2 mM, suggesting that the solute diffusion/adsorption is no longer the limiting-step at this range.

The effect of linear flow velocity and Sc concentration on Sc adsorption of MESG in a fixed bed column is shown in Table 3.

TABLE 3
Effect of linear flow velocity and Sc concentration
on Sc adsorption of MESG in a fixed bed column

| | C₀ | Q | u × 10³ | K_{BA} × 10³ | N₀ | N₀* | | |
|---|---|---|---|---|---|---|---|---|
| Size | (mM) | (mL/min) | (m/s) | (L/mg/min) | (mg/L) | (mg/L) | BP | R² |
| 150-300 | 2.2 | 2.5 | 1.075 | 8.02 | 937.2 | 838.2 | 8.91 | 0.997 |
| 300-500 | 2.2 | 2.5 | 1.075 | 3.22 | 893.1 | 793.4 | 7.73 | 0.977 |
| 500-700 | 2.2 | 2.5 | 1.075 | 1.23 | 889.4 | 790.4 | 5.79 | 0.945 |
| 300-500 | 2.2 | 1.25 | 0.541 | 3.15 | 889.1 | 790.1 | 8.32 | 0.995 |
| 300-500 | 2.2 | 2.5 | 1.075 | 3.22 | 893.1 | 793.4 | 7.73 | 0.977 |
| 300-500 | 2.2 | 5 | 2.16 | 3.62 | 871.6 | 772.6 | 6.52 | 0.978 |
| 300-500 | 2.2 | 9 | 3.90 | 6.77 | 846.9 | 747.9 | 6.35 | 0.947 |
| 300-500 | 2.2 | 2.5 | 1.075 | 3.22 | 893.1 | 793.4 | 7.73 | 0.977 |
| 300-500 | 1.2 | 2.5 | 1.075 | 4.03 | 862.6 | 808.6 | 14.08 | 0.961 |
| 300-500 | 0.56 | 2.5 | 1.075 | 4.60 | 741.0 | 715.8 | 25.84 | 0.979 |
| 300-500 | 0.24 | 2.5 | 1.075 | 4.98 | 717.7 | 706.9 | 58.8 | 0.991 |

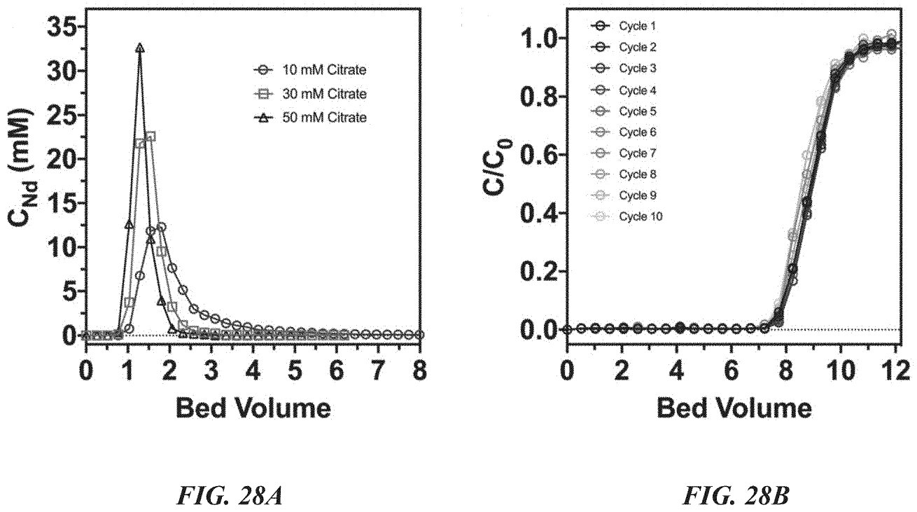

Desorption and column recycle. To recycle MEPG for bed reuse, column desorption experiments were carried out by using pH 6 citrate solutions with different concentrations (10, 30, and 50 mM) at flow rate of 2.5 mL/min. The column was reconditioned by 90 mL of DI-water. As shown in the FIG. 28A, all 3 conditions showed sharp Sc desorption peaks soon after the citrate contacted the column. The concentration of Sc in the desorption solution was also positively related to the concentration of citrate concentrations. For 50 mM citrate, over 95% adsorbed Sc was desorbed within a span of 1 bed volume with the maximum concentration of Sc being 32 mM, which is 14.5 times higher than the 2.2 mM Sc feed solution used for adsorption. The column was also completely regenerated by using 10 mM citrate solution. However, as many as 3 bed volumes of 10 mM citrate solution was required to recycle the adsorbents. These results suggested that the MEPG column can be effectively regenerated using citrate solution with a wide range of concentrations. In addition, the regeneration process was significantly faster than the column breakthrough process especially when feed solution with low Sc concentration was used. Such efficient regeneration allows flexible column operation for real applications.

After a simple wash by DI-water, the column was reused for Sc adsorption to test the column reusability. Sc breakthrough curves were obtained for 10 consecutive adsorption/desorption cycles at a flow rate of 2.5 mL/min. For each cycle, a feed of 2.2 mM Sc in 10 mM pH 3.0 glycine was used for adsorption and 90 mL of 50 mM citrate (pH 6) was used for desorption. As shown in FIG. 28B, almost identical breakthrough curves were observed after 10 cycles of adsorption/desorption experiments, indicating that the MEPG adsorbent could be used in multiple cycles without visibly reducing the adsorption capacity. SEM. TEM and confocal microscope also confirmed that the MEPG structure was unaffected by 10 adsorption/desorption cycles.

From the foregoing, it will be appreciated that specific embodiments of the invention have been described herein for purposes of illustration, but that various modifications may be made without deviating from the scope of the invention. Accordingly, the invention is not limited except as by the appended claims.

Unless the context indicates otherwise, it is specifically intended that the various features of the invention described herein can be used in any combination. Moreover, the disclosure also contemplates that in some embodiments any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Para A. A method for preferentially separating scandium (Sc) from a rare earth element (REE) containing material comprising the steps of: (a) contacting microbes with the REE containing material at a pH between about 3 to about 4 to form Sc-microbe complexes; and (b) separating the Sc from the microbes by contacting the Sc-microbe complexes with a solution comprising an organic chelator, wherein the microbes are Arthrobacter nicotianae (A. nicotianae) microbes.

Para B. The method of Para A, wherein the organic chelator is citrate.

Para C. The method of Para A or B, wherein the solution comprising the organic chelator has a pH of about 5 to about 6.

Para D. The method of any one of Para A-C, wherein in the contacting step (a) Sc is selectively absorbed by the microbes to form the Sc-microbe complexes and the microbes absorb substantially no other REEs, non-REE components, or any other elements in the REE containing material other than Sc.

Para E. The method of any one of Para A-D, wherein the pH of the REE containing material is incrementally adjusted from a pH of about 3 to about 4 in the contacting step (a).

Para F. The method of any one of Para A-E, wherein the pH of the REE containing material is incrementally adjusted from 3 to 3.4, 3.4 to 3.6, and 3.6 to 3.8 in the contacting step (a).

Para G. The method of any one of Para A-F, wherein the solution is incrementally adjusted from pH 5 to 6 in the separating step (b).

Para H. The method of Para G, wherein the other REEs are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and yttrium (Y).

Para I. The method of Para G, wherein the non-REE component is a metal selected from the group consisting of iron (Fe), calcium (Ca), aluminum (Al), magnesium (Mg), zinc (Zn), nickel (Ni), sodium (Na), lithium (Li), potassium (K), cobalt (Co), manganese (Mn), and copper (Cu).

Para J. The method of Para G, wherein the non-REE component is a radionucleotide selected from the group consisting of uranyl (U) and thorium (Th).

Para K. The method of any one of Para A-J, wherein Sc is preferentially separated from Fe in the REE containing material.

Para L. The method of any one of Para A-K, further comprising repeating steps (a) and (b) with a second, third, fourth, fifth, six, seventh, eighth, ninth, tenth or more REE containing material.

Para M. The method of any one of Para A-L, wherein step (b) is repeated until at least about 100%, at least about 90%, at least about 80%, at least about 70%, at least about 60%, at least about 50%, at least about 40%, at least about 30%, at least about 20%, or at least about 10% of the Sc is separated from the Sc-microbe complexes.

Para N. The method of any one of Para A-M, wherein the Sc is separated relative to any other REE, any non-REE component, and/or to any other element in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE, any non-REE component, or any other element.

Para M. The method of any one of Para A-N, wherein the microbes are embedded into a solid support.

Para N. The method of any one of Para A-M, wherein the microbes are embedded into silicon dioxide (SiO₂), polyethylene glycol diacrylate, agarose, and/or acrylamide.

Para O. The method of Para N, wherein a cell density of the microbes in the SiO₂ is about 1 g/ml.

Para P. The method of Para N, wherein a cell density of the microbes in the SiO₂ is about 2 g/ml.

Para Q. The method of any one of Para A-P, further comprising adding the microbes to a column prior to step (a).

Para R. The method of any one of Para A-Q, wherein Fe and/or Al are present in the REE containing material in a concentration three orders of magnitude higher than that of a concentration of Sc.

Para S. The method of any one of Para A-R, wherein the solution comprises citrate.

Para T. The method of any one of Para A-S, wherein the solution comprises citrate at a concentration of about 25 mM.

Para U. The method of any one of Para A-T, wherein the microbes selectively bind to the Sc due to a stronger ionic interaction of Sc relative to other REEs or non-REE components.

Para V. A method for preparing a particle for scandium (Sc) separation from rare earth element (REE) containing material comprising the steps of: (a) encapsulating Arthrobacter nicotianae (A. nicotianae) microbes in a nanoparticle to from microbe encapsulated particles; (b) selecting microbe encapsulated particles having an average size of about 150 μm to about 300 μm, and wherein the microbes are embedded within or on a surface of the particles.

Para W. The method of Para V, wherein the nanoparticle is a silica nanoparticle.

Para X. The method of Para V or W, wherein the encapsulating step (a) includes a condensation reaction of SNPS with hydrolyzed tetraethyl orthosilicate (TEOS) to form a microbe encapsulated gel.

Para Y. The method of any one of Para V-X, wherein prior to step (b), the microbe encapsulated particles are crushed to obtain particles having length in at least one dimension between about 150 μm to about 300 μm.

Para Z. The method of any one of Para V-Y, further comprising incorporating the particle into a column, membrane, bead, or combination thereof.

Para AA. A particle for scandium (Sc) separation comprising Arthrobacter nicotianae (A. nicotianae), wherein the particle has an average pore size of about 50 nm to about 200 nm.

Para AB. The particle of Para AA, wherein the particle has a cuboid shape.

Para AC. The particle of Para AA or AB, wherein the particle has a length in all four dimensions between about 150 μm to about 300 μm.

Para AD. The particle of any one of Para AA-AC, wherein the pore size facilitates the diffusion of REEs into and out of the particle.

Para AE. The particle of any one of Para AA-AD, wherein the pore size prevents the diffusion of A. nicotianae cocci having an average diameter of at least 1 μm from diffusing into and out of the particle.

Para AF. The particle of any one of Para AA-AE, wherein the particle has an A. nicotianae cell density of 1 g/ml.

Para AG. The particle of Para AF, wherein the A. nicotianae cell density is at least about 20 wt % or more of the total weight of the particle or at least about 20 vol % or more of the total volume of the particle.

Para AH. A method for preferentially separating scandium (Sc) and total REEs from a REE containing material comprising the steps of: (a) contacting microbes embedded within a first solid support with the REE containing material at a pH of about 3 to about 4 to form Sc-microbe complexes; (b) collecting the REE containing material, wherein the REE material contains substantially no Sc after contact with the microbes embedded within the first solid support; and (c) contacting microbes embedded within a second solid support with REE material containing substantially no Sc to form REE-microbe complexes.

Para AI. The method of Para AH, wherein prior to the collecting step (b), Sc is separated from the microbes by contacting the Sc-microbe complex with a solution comprising an organic chelator.

Para AJ. The method of Para Al, wherein the organic chelator is citrate.

Para AK. The method of Para Al or AJ, wherein the solution has a pH of 6.

Para AL. The method of any one of Para AH-AK, wherein after the contacting step (c), the total REEs are separated from the microbes by contacting the REE-microbe complexes with solution comprises hydrochloric acid (HCl).

Para AM. The method of Para AL, wherein the solution has a pH of 1.

Para AN. The method of any one of Para AH-AM, wherein prior to the contacting step (c), the pH of the REE containing material containing substantially no Sc is adjusted to about 5 to precipitate non-REE components from the REE containing material, wherein the precipitated non-REEs are filtered from the REE containing material.

Para AO. The method of Para AN, wherein the non-REE components are iron (Fe), aluminum (Al), or both.

Para AP. The method of any one of Para AH-AO, wherein the microbes are Arthrobacter nicotianae (A. nicotianae).

Para AQ. The method of any one of Para AH-AP, wherein Sc is separated from REEs are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and yttrium (Y).

Para AR. A method for preferentially separating one or more rare earth elements (REEs) from an REE containing material comprising the steps of: (a) contacting microbes with the REE containing material to form REE-microbe complexes, wherein the microbes are encapsulated in a polyethylene glycol diacrylate hydrogel; and (b) separating the one or more REEs from the microbes by contacting the REE-microbe complexes with a solution comprising an organic chelator.

Para AS. The method of Para AR wherein the one or more REEs are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), and Scandium (Sc).

Para AT. The method of AR or AS, wherein the one or more REEs is Sc.

Para AU. The method of any one of Para AR-AT, wherein the polyethylene glycol diacrylate hydrogel encapsulated microbes are in a form of a nanoparticle having a having an average size of about 150 μm to about 700 μm.

Para AV. The method of Para AU, wherein the average size is about 300 μm to about 500 μm.

Para AW. The method of Para AU, wherein the average size is about 150 μm to about 300 μm.

Para AX. The method of Para AU, wherein the average size is about 500 μm to about 700 μm.

Para AY. The method of any one of Para AR-AX, further comprising adding the microbes to a column prior to step (a).

Para AZ. The method of any one of Para AR-AY, wherein contacting the microbes with the REE containing material comprises introducing the REE containing material to the column at a flow rate of about 2×10⁻³ m/s to 4×10⁻³ meters per second (m/s).

Para BA. The method of any one of Para AR-AY, wherein the REE containing material comprises the one or more REEs at a concentration of about 1.0 mM to about 3.0 mM.

Para BB. The method of Para BA, wherein the concentration is about 2.2 mM.

Para BC. The method of any one of Para AR-BB, wherein the organic chelator is citrate.

Para BD. The method of any one of Para AR-BC, wherein the solution comprising the organic chelator has a pH of about 5 to about 6.

Para BE. The method of any one of Para AR-BD, wherein the one or more REEs is Sc and in the contacting step (a) Sc is selectively absorbed by the microbes to form the Sc-microbe complexes and the microbes absorb substantially no other REEs, non-REE components, or any other elements in the REE containing material other than Sc.

Para BF. The method of any one of Para AR-BE, wherein a pH of the REE containing material is incrementally adjusted from a pH of about 3 to about 4 in the contacting step (a).

Para BG. The method of any one of Para AR-BF, wherein a pH of the REE containing material is incrementally adjusted from 3 to 3.4, 3.4 to 3.6, and 3.6 to 3.8 in the contacting step (a).

Para BH. The method of any one of Para AR-BG, wherein the solution is incrementally adjusted from pH 5 to 6 in the separating step (b).

Para BI. The method of Para BE, wherein the non-REE component is a metal selected from the group consisting of iron (Fe), calcium (Ca), aluminum (Al), magnesium (Mg), zinc (Zn), nickel (Ni), sodium (Na), lithium (Li), potassium (K), cobalt (Co), manganese (Mn), and copper (Cu).

Para BJ. The method of Para BE, wherein the non-REE component is a radionucleotide selected from the group consisting of uranyl (U) and thorium (Th).

Para BK. The method of any one of Para AR-BJ, further comprising repeating steps (a) and (b) with a second, third, fourth, fifth, six, seventh, eighth, ninth, tenth or more REE containing material.

Para BL. The method of any one of Para AR-BJ, wherein step (b) is repeated until at least about 100%, at least about 90%, at least about 80%, at least about 70%, at least about 60%, at least about 50%, at least about 40%, at least about 30%, at least about 20%, or at least about 10% of the one or more REEs is separated from the REE-microbe complexes.

Para BM. The method of any one of Para AR-BL, wherein the one or more REEs is separated relative to any other REE, any non-REE component, and/or to any other element in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE, any non-REE component, or any other element.

Para BN. The method of any one of Para AR-BM, wherein the solution comprises citrate.

Para BO. The method of any one of Para AR-BN, wherein the solution comprises citrate at a concentration of about 25 mM.

Para BP. A method for preparing a particle for separation one or more rare earth elements (REEs) from REE containing material comprising the steps of: (a) encapsulating microbes in a polyethylene glycol diacrylate hydrogel to from microbe encapsulated particles; and (b) (b) selecting microbe encapsulated particles having an average size of about 300 μm to about 500 μm, wherein the microbes are embedded within or on a surface of the particles.

Para BQ. The method of Para BP, wherein the microbes are encapsulated in a polyethylene glycol diacrylate hydrogel by free radical polymerization of polyethylene glycol diacrylate.

Para BR. The method of Para BP or BQ, wherein the one or more REEs are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), and Scandium (Sc).

Para BS. The method of any one of Para BP-BR, wherein the one or more REEs is Sc.

Para BT. The method of any one of Para BP-BR, wherein prior to step (b), the microbe encapsulated particles are crushed to obtain particles having an average size of about 150 μm to about 700 μm.

Para BQ. The method of any one of Para BP-BT, further comprising selecting microbe encapsulated particles having an average size of about 300 μm to about 500 μm from the particles having an average size of about 150 μm to about 700 μm.

Para BR. The method of any one of Para BP-BQ, wherein the particle has a cuboid shape.

Para BS. The method of any one of Para BP-BR, wherein the particle has an A. nicotianae cell density of 1 g/ml.

Para BT. The method of any one of Para BP-BS, wherein an A. nicotianae cell density is at least about 20 wt % or more of the total weight of the particle or at least about 20 vol % or more of the total volume of the particle.

Para BU. The method of any one of Para BP-BT, wherein the one or more REEs are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), yttrium (Y), and Scandium (Sc).

Para BV. The method of any one of Para BP-BU, wherein the one or more REEs is Sc.

Para BW. A method for preferentially separating scandium (Sc) from a REE containing material comprising the steps of: (a) adding microbes embedded within polyethylene glycol diacrylate hydrogel to a column; (b) introducing to the microbes embedded within polyethylene glycol diacrylate hydrogel the REE containing material at a flow rate of about 2×10⁻³ m/s to 4×10⁻³ meters per second (m/s) and at a pH of about 3 to about 4 to form Sc-microbe complexes; and (c) separating the Sc from the microbes by contacting the Sc-microbe complexes with a solution comprising an organic chelator.

Para BX. The method of Para BW, wherein Sc is present in the REE containing material at a concentration of about 1 μM to about 3 mM.

Para BY. The method of Para BW or BX, wherein Sc is present in the REE containing material at a concentration of about 2 mM.

Para BZ. The method of any one of Para BW-BY, wherein the organic chelator is citrate.

Para CA. The method of any one of Para BW-BZ, wherein the solution has a pH of about 6.

Para CB. The method of any one of Para BW-CA, wherein the microbes are Arthrobacter nicotianae (A. nicotianae).

Para CD. The method of any one of Para BW-CB, wherein Sc is separated from REEs are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and yttrium (Y).

### REFERENCES

1. Brewer, A., et al., Recovery of Rare Earth Elements from Geothermal Fluids through Bacterial Cell Surface Adsorption. Environmental Science & Technology, 2019. 53(13): p. 7714-7723.
2. Brewer, A., et al., Microbe Encapsulation for Selective Rare-Earth Recovery from Electronic Waste Leachates. Environ Sci Technol, 2019.
3. Park, D. M., et al., Recovery of Rare Earth Elements from Low-Grade Feedstock Leachates Using Engineered Bacteria. Environ Sci Technol, 2017. 51(22): p. 13471-13480.
4. Park, D., et al., A Biosorption-Based Approach For Selective Extraction Of Rare Earth Elements From Coal Byproducts. Separation and Purification Technology, 2020. 241: p. 116726.
5. Ku, H. H., Notes On The Use Of Propagation Of Error Formulas. Journal of Research of the National Bureau of Standards—C. Engineering and Instrumentation, 1966. 70C(4): p. 263-273.
6. Mutlu, B. R., et al., Silicon Alkoxide Cross-Linked Silica Nanoparticle Gels For Encapsulation Of Bacterial Biocatalysts. Journal of Materials Chemistry A, 2013. 1(36): p. 11051-11060.
7. D'Angelo, P., et al., Hydration Properties and Ionic Radii of Actinide(III) Ions in Aqueous Solution. Inorganic Chemistry, 2013. 52(18): p. 10318-10324.
8. Müller, G. T. A. and R. H. Stokes, The Mobility Of The Undissociated Citric Acid Molecule In Aqueous Solution. Transactions of the Faraday Society, 1957. 53(0): p. 642-645.
9. Jones, D. and R. M. Keddie, The Genus Arthrobacter, in The Prokaryotes: Volume 3: Archaea. Bacteria: Firmicutes, Actinomycetes, M. Dworkin, et al., Editors. 2006, Springer New York: New York, N.Y. p. 945-960.
10. Qi, D., Chapter 7—Chemical Separation Method, in Hydrometallurgy of Rare Earths, D. Qi, Editor. 2018, Elsevier. p. 671-741.
11. Zhang, W. Z. et al., Efficient and Selective Recovery of Trace Scandium by Inorganic Titanium Phosphate Ion-Exchangers from Leachates of Waste Bauxite Residue. Acs Sustain Chem Eng 2017, 5 (4), 3103-3114.
12. Roosen, J., et al., Recovery of scandium from leachates of Greek bauxite residue by adsorption on functionalized chitosan-silica hybrid materials. Green Chem 2016, 18 (7), 2005-2013.
13. Park, D., et al., A biosorption-based approach for selective extraction of rare earth elements from coal byproducts. Separation and Purification Technology 2020, 241, 116726.
14. Bauer, D.; Diamond, D.; Li, J.; Sandalow, D.; Telleen, P.; Wanner, B. U.S. Department of Energy Critical Materials Strategy; None; TRN: US201104%%69 United States 10.2172/1000846 TRN: US201104%%69 DOEEE English; None: 2010; p Medium: ED.
15. Cotton, S. A., Scandium, Yttrium & the Lanthanides: Inorganic & Coordination Chemistry. In Encyclopedia of Inorganic and Bioinorganic Chemistry.
16. Moriwaki, H.; et al., Application of Freeze-Dried Powders of Genetically Engineered Microbial Strains as Adsorbents for Rare Earth Metal Ions. Acs Appl Mater Inter 2016, 8 (40), 26524-26531.
17. Giret, S., et al., Selective Separation and Preconcentration of Scandium with Mesoporous Silica. Acs Appl Mater Inter 2018, 10 (1), 448-457.
18. Zhao, Z. G., et al., Development of novel adsorbent bearing aminocarbonylmethylglycine and its application to scandium separation. J Chem Technol Biot 2016, 91 (11), 2779-2784.
19. Thapa, R., et al., Bisphosphonate modified mesoporous silicon for scandium adsorption. Micropor Mesopor Mat 2020, 296.
20. Avdibegovic, D., et al., Efficient separation of rare earths recovered by a supported ionic liquidfrom bauxite residue leachate. Rsc Adv 2018, 8 (22), 11886-11893.
21. Avdibegovic, D., et al., Recovery of scandium(III) from diluted aqueous solutions by a supported ionic liquid phase (SILP). Rsc Adv 2017, 7 (78), 49664-49674.
22. Wang, W. W., et al., Metallurgical processes for scandium recovery from various resources: A review. Hydrometallurgy 2011, 108 (1-2), 100-108.

I/We claim:

1. A method for preferentially separating scandium (Sc) from a rare earth element (REE) containing material comprising the steps of:
(a) contacting microbes with the REE containing material at a pH between about 3 to about 4 to form Sc-microbe complexes; and
(b) separating the Sc from the microbes by contacting the Sc-microbe complexes with a solution comprising an organic chelator,
wherein the microbes are Arthrobacter nicotianae (A. nicotianae) microbes.

2. The method of claim 1, wherein the organic chelator is citrate.

3. The method of claim 1, wherein the solution comprising the organic chelator has a pH of about 5 to about 6.

4. The method of claim 1, wherein in the contacting step (a) Sc is selectively absorbed by the microbes to form the Sc-microbe complexes and the microbes absorb substantially no other REEs, non-REE components, or any other elements in the REE containing material other than Sc.

5. The method of claim 1, wherein the pH of the REE containing material is incrementally adjusted from a pH of about 3 to about 4 in the contacting step (a).

6. The method of claim 1, wherein the pH of the REE containing material is incrementally adjusted from 3 to 3.4, 3.4 to 3.6, and 3.6 to 3.8 in the contacting step (a).

7. The method of claim 1, wherein the solution is incrementally adjusted from pH 5 to 6 in the separating step (b).

8. The method of claim 4, wherein the other REEs are selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and yttrium (Y).

9. The method of claim 4, wherein the non-REE component is a metal selected from the group consisting of iron (Fe), calcium (Ca), aluminum (Al), magnesium (Mg), zinc (Zn), nickel (Ni), sodium (Na), lithium (Li), potassium (K), cobalt (Co), manganese (Mn), and copper (Cu).

10. The method of claim 4, wherein the non-REE component is a radionucleotide selected from the group consisting of uranyl (U) and thorium (in).

11. The method of claim 1, wherein Sc is preferentially separated from Fe in the REE containing material.

12. The method of claim 1, further comprising repeating steps (a) and (b) with a second, third, fourth, fifth, six, seventh, eighth, ninth, tenth or more REE containing material.

13. The method of claim 1, wherein step (b) is repeated until at least about 100%, at least about 90%, at least about 80%, at least about 70%, at least about 60%, at least about 50%, at least about 40%, at least about 30%, at least about 20%, or at least about 10% of the Sc is separated from the Sc-microbe complexes.

14. The method of claim 1, wherein the Sc is separated relative to any other REE, any non-REE component, and/or to any other element in a purity of at least about 10%, at least about 15%, at least about 20%, at least about 30%, at least about 40%, at least about 50%, at least about 55%, at least about 60%, at least about 65%, at least about 70%, at least about 80%, at least about 85%, at least about 90%, at least about 95%, or at least about 100%, relative to any other REE, any non-REE component, or any other element.

15. The method of claim 1, wherein the microbes are embedded into a solid support.

16. The method of claim 1, wherein the microbes are embedded into silicon dioxide (SiO₂), polyethylene glycol diacrylate, agarose, and/or acrylamide.

17. The method of claim 15, wherein a cell density of the microbes in the SiO₂ is about 1 g/ml.

18. The method of claim 15, wherein a cell density of the microbes in the SiO₂ is about 2 g/ml.

19. The method of claim 1, further comprising adding the microbes to a column prior to step (a).

20. The method of claim 1, wherein Fe and/or Al are present in the REE containing material in a concentration three orders of magnitude higher than that of a concentration of Sc.

21. The method of claim 1, wherein the solution comprises citrate.

22. The method of claim 1, wherein the solution comprises citrate at a concentration of about 25 mM.

23. The method of claim 1, wherein the microbes selectively bind to the Sc due to a stronger ionic interaction of Sc relative to other REEs or non-REE components.
